(19) United States
(12) Patent Application Publication
(10) Pub. No.: US 2026/0325871 A1
(43) Pub. Date: Oct. 22, 2026

(54) BIOLOGICALLY-GUIDED MONITORING AND TREATMENT ASSISTANCE SYSTEM

(71) Applicant: Alejandro ACHKIENASI AMEZCUA, Be'er Sheva (IL)

(72) Inventor: Alejandro ACHKIENASI AMEZCUA, Be'er Sheva (IL)

(73) Assignee: Omega Radiant LTD, Be'er Sheva (IL)

(21) Appl. No.: 19/653,487

(22) Filed: Apr. 21, 2026

Related U.S. Application Data
(60) Provisional application No. 63/792,763, filed on Apr. 22, 2025.

(51) Int. Cl. A61N 5/10; A61B 5/0536; A61B 5/0538; A61B 5/273

(52) U.S. Cl. CPC A61N 5/103; A61B 5/0536; A61B 5/0538; A61B 5/273; A61N 2005/1097

(57) ABSTRACT

A biologically-guided monitoring and treatment assistance system comprises a data interface configured to receive signals from a plurality of radiolucent electrodes; a surface detection module configured to identify a spatial orientation of said plurality of radiolucent electrodes relative to a target anatomical region of a patient; an impedance acquisition unit coupled to said plurality of radiolucent electrodes; and a processing unit. The processing unit is configured to generate a spatial co-registration of the spatial orientation from the surface detection module with anatomical imaging data of the target anatomical region to define a digital volumetric mesh; reconstruct at least one volumetric conductivity map from data provided by the impedance acquisition unit by mapping said data onto the volumetric mesh; and superimpose said volumetric conductivity map onto the anatomical imaging data to identify one or more biological responses.

### CROSS-REFERENCES TO RELATED APPLICATIONS

This application claims priority of the US provisional Application No. 63/792,763 filed on Apr. 22, 2025, entitled BIOLOGICALLY-GUIDED ADAPTIVE RADIATION THERAPY SYSTEM USING REAL-TIME ELECTRICAL IMPEDANCE TOMOGRAPHY ENHANCED BY ANATOMICAL IMAGING MODALITIES FOR IMAGE QUALITY IMPROVEMENT AND INTEGRATED ELECTRODE IMMOBILIZATION by Alejandro ACHKIENASI AMEZCUA.

### TECHNICAL FIELD

The present invention relates generally to the fields of medical imaging, bioelectrical monitoring, and treatment assistance. More specifically, the present invention pertains to systems and methods for biologically-guided monitoring that utilize electrical impedance tomography (EIT) data integrated with anatomical imaging data to identify and monitor biological responses in a target anatomical region of a patient.

### BACKGROUND

Electrical Impedance Tomography (EIT) is a non-invasive, non-ionizing imaging technique that measures the internal electrical properties of a subject—such as conductivity, impedance, and permittivity—to generate volumetric images of biological tissues. The standard EIT workflow typically involves: (i) the placement of multiple electrodes around a target anatomical region; (ii) the acquisition of impedance measurements by applying small electrical currents and recording the resulting surface voltages; and (iii) the use of reconstruction algorithms to map these measurements into a spatial representation of the tissue's electrical characteristics.

The advantages of EIT include its real-time imaging capability, portability, and lack of ionizing radiation exposure, making it suitable for continuous bedside monitoring. However, conventional EIT faces significant technical hurdles that have limited its clinical adoption, particularly in high-precision medical environments. These include relatively low spatial resolution, extreme sensitivity to electrode positioning (where even sub-millimeter shifts can induce significant image artifacts), and the mathematical complexity of managing non-linear electrical paths through heterogeneous biological structures.

These limitations are further exacerbated in clinical domains that are highly imaging-dependent, such as radiation therapy. Before the radiation treatment begins, patients typically undergo simulation scans using computed tomography (CT), magnetic resonance imaging (MRI), or both to enable tumor localization, organ-at-risk segmentation, and treatment planning. During the course of therapy, imaging continues to play a critical role in patient positioning and treatment verification. Modalities such as cone-beam CT (CBCT), X-ray fluoroscopy, surface-guided imaging, and thermographic cameras are used to align the patient and track anatomical structures, albeit with important limitations.

For example, fluoroscopy provides continuous X-ray feedback but often requires invasive placement of fiducial markers (e.g., gold seeds), exposes the patient to additional ionizing radiation, and delivers relatively low-contrast images. Surface-guided techniques offer high precision but are limited to external contours and cannot capture internal changes. The most advanced systems—MRI-LINACs—enable high-quality real-time soft tissue imaging, but they remain prohibitively expensive and complex, restricting access to a small subset of institutions.

Despite their ubiquity, none of these imaging methods are capable of visualizing the biological impact of radiation therapy during treatment. The effectiveness of the treatment is typically assessed only months later through follow-up imaging (e.g., PET, MRI, or CT), once the full biological response has manifested. As a result, clinicians currently lack tools to evaluate, during therapy, whether the intended tissues are responding as expected, whether critical structures are being spared, or whether the treatment strategy should be modified based on tissue-specific radiation sensitivity. Without biological feedback, there is no mechanism to adapt the prescribed dose or its spatial distribution according to the actual response of the tumor or surrounding tissues—responses which may vary significantly from patient to patient and may only become evident after multiple treatment fractions. Incorporating such feedback, even intermittently, could enable significantly more effective and personalized radiation therapy compared to current standards.

The emerging field of adaptive radiation therapy (ART) seeks to address some of these challenges. ART involves modifying the treatment plan based on updated anatomical imaging—either in real-time, online, or offline—to account for inter-fraction or intra-fraction anatomical changes. However, current adaptive workflows rely solely on geometric information (e.g., tumor displacement, shrinkage, or deformation) and do not provide insight into the actual biological response of tissues to radiation.

Electrical impedance tomography (EIT) offers a promising alternative. EIT is a non-invasive, non-ionizing imaging modality that provides real-time, volumetric maps of electrical conductivity within biological tissues. Ionizing radiation, when interacting with tissue, initiates a chain of events beginning with atomic ionization and radical formation, followed by chemical bond breakage and biological effects. Each of these stages can result in changes to the electrical conductivity of the affected tissue. For example, ionization causes electron vacancies that alter local charge transport; free radical formation and membrane disruption further modify ionic content and mobility, all of which affect conductivity.

The sensitivity of EIT to these biophysical changes makes it a strong candidate for tracking radiation-induced effects during therapy, not just after. By continuously imaging changes in conductivity, EIT has the potential to act as a real-time surrogate biomarker for treatment effectiveness and biological response. However, EIT has not yet been adopted clinically in radiation therapy due to several significant technical barriers:
Electrode placement reproducibility across treatment fractions is difficult to achieve using traditional adhesive electrodes.
External contouring between electrodes and the patient's surface must be known accurately for reliable EIT reconstruction.
Standard metal electrodes cause artifacts in CT and CBCT imaging and can interfere with dose delivery due to their high atomic number.
Low spatial resolution in EIT images makes interpretation and localization of dose effects challenging.

Additionally, the current concept of dosimetry—measuring the physical dose delivered in Gray (Gy)—does not account for tissue-specific biological sensitivity, radical formation, or individualized cellular response. Current clinical protocols are largely limited by a lack of tools capable of providing non-invasive, real-time biological feedback.

Prior inventions have explored aspects of this integration. For example:
U.S. application Ser. No. 14/687,497 (Real-Time and Non-Invasive In Vivo Dosimetry and Tissue Monitoring Using Electrical Impedance Tomography for Radiation Therapy) describes EIT systems to measure conductivity changes during radiation delivery, primarily for dose localization. However, it does not address biological interpretation, adaptive planning, or the use of multimodal imaging to enhance reconstruction.
U.S. application Ser. No. 13/965,341 (Non-Invasive Location and Tracking of Tumors and Other Tissues for Radiation Therapy) proposes using EIT for intra-treatment tumor localization. Yet, it does not incorporate biological response modeling or electrode immobilization strategies to enable reproducible multi-fraction imaging.

Other filings (e.g., WO/2013/175627 and US20130144191) focus on EIT hardware (electrode belts, circuit protection), without clinical radiotherapy context or biologically driven workflows.

These prior approaches do not address key challenges that prevent clinical deployment of EIT in radiation therapy, including:
Inconsistent electrode placement across fractions, leading to spatial errors.
Image artifacts from standard metal electrodes interfering with CT/CBCT scans and dose delivery.
Lack of integration with existing anatomical imaging for improved boundary modeling and reconstruction accuracy.
Absence of adaptive algorithms to guide clinical decision-making based on dynamic conductivity trends.

There is a need for a monitoring framework that addresses these limitations by providing a biologically-guided monitoring and treatment assistance. It would be advantageous to provide a system that utilizes imaging-compatible electrode interfaces to acquire consistent, high-fidelity conductivity data during and across clinical sessions. By integrating anatomical imaging modalities with real-time bioelectrical signatures, such a system can enhance reconstruction quality, enable co-registration of anatomical and conductivity data, and allow for the visualization of the spatial distribution of biological responses. Such a platform is configured to improve therapeutic precision and enable personalized, biology-driven treatment adaptation across various clinical applications.

The terms ‘diagnostic imaging table,’ ‘patient support,’ and ‘treatment couch’ may be used interchangeably hereinafter.

### SUMMARY OF EMBODIMENTS OF THE INVENTION

According to an aspect of the present invention, there is provided a biologically-guided monitoring and treatment assistance system. The system comprises a data interface configured to receive signals from a plurality of radiolucent electrodes; a surface detection module configured to identify a spatial orientation of said plurality of radiolucent electrodes relative to a target anatomical region of a patient; an impedance acquisition unit coupled to said plurality of radiolucent electrodes; and a processing unit configured to:
(i) generate a spatial co-registration of said spatial orientation from said surface detection module with anatomical imaging data of the target anatomical region to define a digital volumetric mesh;
(ii) reconstruct at least one volumetric conductivity map from data provided by said impedance acquisition unit by mapping said data onto said volumetric mesh; and
(iii) superimpose said volumetric conductivity map onto said anatomical imaging data to identify at least one biological response not characterized by the anatomical imaging data.

According to some embodiments, the system provides a multi-stage data architecture wherein the data interface and the impedance acquisition unit operate as distinct functional entities. For example, the impedance acquisition unit may be configured to perform active excitation and measurement routines via the plurality of radiolucent electrodes, while the data interface serves as a logical or physical gateway to receive, format, and synchronize signals—including raw electrical data, electrode status, or positioning signals—for the processing unit.

In the context of the present disclosure, the term “anatomical imaging data” refers to spatial data representing the internal structural morphology of the patient. This may include, by way of non-limiting example, data acquired via computed tomography (CT), magnetic resonance imaging (MRI), cone-beam CT (CBCT), positron emission tomography (PET), or ultrasound. The “anatomical imaging data” may serve to provide a structural reference frame (e.g., a “prior”) for the electrical impedance tomography (EIT) reconstruction. The scope of the invention is not limited to any specific imaging modality or combination thereof.

The term ‘radiolucent electrodes’ shall denote conductive elements characterized by low attenuation of high-energy radiation beams (e.g., Megavoltage X-rays or protons). Such electrodes may be comprised of carbon-based materials, conductive polymers, or low-atomic-number (low-Z) composites configured to minimize dose perturbation and imaging artifacts. The term ‘radiolucent electrodes’ is not limited to any specific material or carrier, provided the electrodes permit the acquisition of impedance data without substantially interfering with a concurrent therapeutic beam or co-registered imaging modality.

In the context of the present disclosure, the term ‘biologically-guided’ may refer to the use of real-time or near-real-time physiological data to inform, adjust, or verify a clinical intervention.

In the context of the present disclosure, “generating a spatial co-registration” refers to a machine-executed co-registration process that establishes a common coordinate system between the physical space of the patient and the digital space of the anatomical imaging data.
Machine-Driven Co-registration: This process involves the high-speed transformation of surface data (captured by the surface detection module) into a mathematical alignment with three-dimensional voxels of the anatomical imaging. This generation of a spatial co-registration involves the automated resolution of multi-dimensional spatial transforms and point-cloud co-registrations to anchor the impedance measurements to specific internal tissues within a unified coordinate system.
The Digital Volumetric Mesh: The resulting “digital volumetric mesh” is a discrete, multi-dimensional data structure that partitions the target anatomical region into a finite element grid. Each element within this digital mesh is assigned specific electrical and spatial properties derived from the co-registration. This mesh serves as the essential computational scaffold required to solve the ill-posed inverse problem of EIT, transforming raw surface voltages into a reconstructed map of internal conductivity.

More particularly, the generation of the co-registration and the definition of the digital mesh are performed in real-time or near-real-time, enabling the system to account for patient movement or respiratory gating. These steps represent a technical solution to the challenge of mapping functional biological signals onto static structural images, providing a level of spatial precision and computational complexity that exceeds human cognitive capacity.

According to some embodiments the system further includes one or more immobilization interfaces comprising a thermoplastic material and a plurality of radiolucent electrodes electrically coupled to the impedance acquisition unit. The radiolucent electrodes are embedded within the thermoplastic, and wherein at least one of said radiolucent electrodes is coupled to a radiolucent biasing element configured to maintain constant physical contact between the electrode coupled to biasing element and the patient's skin.

In the context of the present disclosure, the term “immobilization interface” refers to any structural element or assembly used to stabilize a portion of the patient's body. This may include, by way of non-limiting example, thermoplastic masks, vacuum-formed cushions, casts, or stereotactic frames. The immobilization interface may further serve to ensure that the spatial relationship between the electrodes and the patient's internal anatomy remains substantially constant during or between treatments.

In some embodiments the processing unit is further configured to synchronize reconstruction of one or more volumetric conductivity map with a radiation delivery schedule of a radiotherapy treatment beam.

In the context of the present disclosure, the term “synchronize” refers to the temporal or functional coordination between the reconstruction of the volumetric conductivity map and an external event or schedule, such as a radiation delivery sequence.

This synchronization may include, by way of non-limiting example:
Temporal Gating: Configuring the processing unit to acquire or process data during specific windows, such as during active beam-on periods or during interleaved beam-off pulses to minimize signal interference.
Triggered Activation: Initiating data acquisition or reconstruction algorithms in response to a signal from a therapeutic delivery system indicating the commencement or cessation of a dose fraction.
Time-Stamping and Correlation: Mapping reconstructed conductivity shifts to specific timestamps within a treatment log to enable the correlation of localized biological responses with specific doses of delivered energy.

Synchronization may serve to associate the captured biological response with specific therapeutic triggers that induced the response, although the system may also be configured for asynchronous or continuous data acquisition and correlation.

In some examples the processing unit includes a forward modeling module configured to generate a patient-specific conductivity distribution using segmented anatomical imaging data and organ-specific conductivity values. Additionally, the processing unit is configured to perform an inverse reconstruction of EIT data using the forward modeling module as an anatomical prior to generate a patient-specific conductivity baseline derived from a pre-treatment measurement; and subsequent volumetric conductivity maps during or between one or more treatment fractions. The processing unit determines a conductivity change by comparing the subsequent maps to the patient-specific conductivity baseline.

In the context of the present disclosure, the “forward modeling module” may serve to simulate the expected electrical behavior of a specific patient's anatomy based on known structural data. Such known structural data may comprise the spatial coordinates of tissue boundaries, organ volumes, or material interfaces derived from anatomical imaging or a standardized anatomical atlas.

The “inverse reconstruction” may utilize this forward model as an “anatomical prior”—providing an initial spatial constraint to refine the interpretation of the measured EIT signals. By comparing real-time measurements against a “patient-specific conductivity baseline,” the system may isolate transient changes in tissue conductivity (e.g., biological responses) from the static background anatomy. This comparative approach may improve spatial resolution and reduce artifacts, though the system may also utilize non-comparative or generalized reconstruction techniques.

In some embodiments exampling the invention, the processing unit includes an AI-based module is configured to translate reconstructed EIT images into synthetic anatomical representations using a trained image-to-image deep learning model.

In the context of the present disclosure, the “AI-based module” may serve to transform volumetric conductivity data into a visual format that is spatially and structurally familiar to a clinician. This “synthetic anatomical representation” may comprise a generated image that simulates the appearance of a structural scan (e.g., a “synthetic CT”) while being derived from the functional signatures captured by EIT.

This may serve several practical clinical purposes, including:
Real-Time Morphological Verification: Providing a visual confirmation of organ boundaries or tumor displacement during treatment without the use of additional ionizing radiation (e.g., “radiation-free” CBCT).
Cross-Modal Co-Registration: Mapping functional conductivity shifts onto a high-contrast structural template to assist in the precise localization of a biological response.
Enhanced Interpretability: Converting abstract impedance meshes into recognizable tissue contours to facilitate rapid clinical decision-making during or between treatment fractions.

It is to be understood that the “trained image-to-image deep learning model” may utilize a neural network (e.g., a Generative Adversarial Network or a Convolutional Neural Network) trained on paired datasets of EIT and anatomical imaging to learn the mapping between bioelectrical signatures and structural morphology.

In some embodiments the processing unit is further configured to store a plurality of temporal reconstructed volumetric conductivity maps and identify temporal trends in biological response by comparing conductivity values across said plurality of reconstructed volumetric conductivity maps.

In the context of the present disclosure, the longitudinal analysis via temporal reconstructed volumetric conductivity maps may comprise data acquired at different time points, such as before, during, or after a specific treatment fraction, or across a multi-week course of therapy.

The identification of “temporal trends” may serve to monitor the progression or regression of a biological response. This longitudinal analysis may include, by way of non-limiting example:
Adaptive Response Tracking: Identifying deviations from a dielectric baseline within a tumor that may indicate a change in cellular density, oxygenation, or treatment sensitivity.
Early Toxicity Detection: Detecting cumulative changes in the conductivity of organs-at-risk (OARs) that may serve as a precursor to radiation-induced inflammation or fibrosis.
Sub-Clinical Collateral Damage Monitoring: Identifying early bioelectrical indicators of stress or injury in healthy tissues adjacent to the target volume, which may precede detectable anatomical complications or clinical symptoms.
Fraction-to-Fraction Verification: Comparing current maps to a plurality of previous maps to ensure the physiological signature remains consistent with the intended treatment plan.

It is to be understood that the processing unit may utilize statistical analysis, machine learning, or delta-conferencing algorithms to isolate these temporal trends from transient noise or daily anatomical variations.

In some cases at least one of the maps is reconstructed following ionizing radiation delivery and the identification of the temporal trends associated with conductivity changes induced by the ionizing radiation delivery.

At the Bragg peak, the Linear Energy Transfer (LET) of protons or heavy ions increases sharply as the particles reach the end of their range. This rapid energy deposition may cause localized disruption to cellular membranes and ionic environments, creating a detectable signature or “spike” in the volumetric conductivity map.

The processing unit may be further configured to verify the beam range of a particle therapy beam by identifying a localized increase in conductivity corresponding to a Bragg peak location.

In some examples, the system may support Bragg peak range estimation by quantifying conductivity changes along the path of particle deposition, particularly near the distal end of the ion track where biological impact is maximized. The processing unit may be configured to visualize these features in real time or flag deviations from expected patterns to assist in adaptive treatment workflows. Such Bragg peak validation may enable range tracking to ensure the peak is localized within the intended target volume, thereby minimizing dose to adjacent healthy tissues.

More particularly, identifying a “localized increase in conductivity corresponding to a Bragg peak location” may refer to the detection of a concentrated zone of ionization or thermal energy within the target anatomical region.

This verification of the beam range of a particle therapy beam may be performed by way of non-limiting example through:
Ionization Density Mapping: Detecting the spike in conductivity caused by the dense cloud of ions and free radicals generated at the end of the particle's range.
Transient Thermal Detection: Identifying localized changes in the dielectric properties of tissue resulting from the instantaneous “micro-heating” effect at the point of maximum energy deposition.
Spatial Correlation: Comparing the identified peak location against the planned coordinates in the treatment software to verify that the particle beam is stopping exactly where intended.

It is to be understood that the identification of the Bragg Peak may serve to provide real-time “range verification,” allowing for the immediate adjustment or cessation of the particle therapy beam if a deviation from the treatment plan is detected.

The processing unit may be configured to generate clinical alerts when a change in one or more volumetric conductivity maps exceeds a predefined threshold.

In the context of the present disclosure, the “clinical alert” may serve as an automated notification to a practitioner that a measured biological response has deviated from an expected physiological range. This may include, by way of non-limiting example:
Toxicity and Collateral Damage Alerts: Notifying a clinician if the conductivity of an organ-at-risk (OAR) shifts beyond a predefined threshold, which may indicate early-stage radiation-induced inflammation, edema, or acute cellular stress.
Pathological Identification Alerts: Flagging the presence of regions with anomalous conductivity signatures that may correspond to defective or degenerated cells, such as malignant tissue or plaques associated with neurodegenerative pathologies (e.g., Alzheimer's or Dementia).
Treatment Efficacy Alerts: Generating an alert if the target tissue (e.g., a tumor) fails to show a predicted change in conductivity during a treatment session, potentially indicating radio-resistance or an under-dose.

The “predefined threshold” may be a dynamic value based on the patient's own baseline or a statistical population model. The clinical alert may be provided in real-time during a therapy session to assist in adaptive workflows, or generated as part of a post-treatment review to inform long-term care decisions.

The processing unit may export data derived from the volumetric conductivity maps to a treatment planning system (TPS) for recalculation of a radiation dose.

In some embodiments the system includes a patient support assembly having a tabletop, where at least a portion of this tabletop is radiolucent and includes radiolucent electrodes.

In some example embodying the invention, the system includes at least one internal electrode integrated into a catheter needle or a brachytherapy applicator, where the processing unit is configured to reconstruct the volumetric conductivity maps using electrical impedance measurements generated between the internal electrode and at least one of the radiolucent electrodes.

In the context of the present disclosure, an “internal electrode” refers to a conductive element configured for temporary or permanent placement within a subject's body. This may include, by way of non-limiting example, the integration of a conductive tip or shaft into a catheter needle, a biopsy needle, or a specialized brachytherapy applicator. The use of an internal electrode may serve to:
Enhance Localized Sensitivity: Providing a high-resolution “near-field” impedance measurement directly within or adjacent to a target volume (e.g., a tumor core), which may be used to calibrate or “anchor” the volumetric conductivity maps.
Enable Interstitial Monitoring: Capturing real-time biological responses during invasive procedures, such as the placement of radioactive seeds or the delivery of liquid pharmaceuticals, where the needle itself serves a dual purpose as both a delivery vehicle and a diagnostic sensor.
Improve Signal-to-Noise Ratio: Reducing the electrical path length between electrodes, thereby potentially increasing the sensitivity of the system to minute metabolic or ionic shifts that might be attenuated in purely surface-based measurements.

The catheter needle may be a monopolar or bipolar electrode, where the needle body is insulated except at specific active sites to ensure that the impedance measurements are precisely localized to the intended anatomical region.

A method according to some aspects of the present invention for biologically-guided monitoring and treatment assistance includes:
receiving, at a processing unit, impedance data originating from radiolucent electrodes;
identifying, via a surface detection module, a spatial orientation of the radiolucent electrodes relative to a target anatomical region of a patient;
collecting impedance data from the target anatomical region via an impedance acquisition unit coupled to the radiolucent electrodes;
generating, via a processing unit, a spatial co-registration of said spatial orientation with anatomical imaging data of the target anatomical region to define a digital volumetric mesh;
reconstructing at least one volumetric conductivity map by mapping said collected impedance data onto the volumetric mesh; and
identifying at least one biological response not characterized by the anatomical imaging data by superimposing said at least one volumetric conductivity map onto the anatomical imaging data.

The method may include securing at least one immobilization interface comprising said plurality of radiolucent electrodes to the patient.

In some embodiments the method includes synchronizing, via the processing unit, the reconstruction of said at least one volumetric conductivity map with a radiation delivery schedule of a radiotherapy treatment beam.

According to some embodiments the method includes:
generating, via the processing unit, a patient-specific initial conductivity distribution using a forward modeling module and segmented anatomical imaging data with organ-specific conductivity values;
performing, via the processing unit, an inverse reconstruction of EIT data using the forward modeling module as an anatomical prior to incorporate anatomical imaging data as spatial and electrical priors; and
generating, via the processing unit, a patient-specific conductivity baseline using a baseline conductivity model derived from the anatomical imaging data and organ-specific conductivity references. Said reconstructing includes generating subsequent volumetric conductivity maps and comparing them to said patient-specific conductivity baseline.

In the context of the present disclosure, the generation of a “patient-specific initial conductivity distribution” and the subsequent “inverse reconstruction” represent a “multi-stage” computational framework for mapping internal physiological states.

### (a) Forward Modeling as a Structural Constraint

The forward modeling module utilizes segmented anatomical imaging data (e.g., from CT, MRI, or anatomical atlases) to define the spatial boundaries and expected electrical properties of internal structures. By assigning organ-specific conductivity values to these segments, the system generates a simulated electrical baseline. This serves as a “mathematical simulation” or an idealized reference that represents the predicted electrical behavior of the subject's anatomy prior to the integration of real-time measurements.

### (b) Inverse Reconstruction using Anatomical Priors

The “inverse reconstruction” phase incorporates active EIT measurements acquired from the subject. Given that EIT reconstruction is an “ill-posed” inverse problem, the system utilizes the forward model from step (a) as an “anatomical prior.” This prior functions as a spatial and electrical constraint, guiding the reconstruction algorithm to prioritize solutions that are anatomically plausible. Rather than calculating conductivity from a null state, the processing unit iteratively refines the initial distribution to identify deviations between the simulated model and the real-world captured data.

### (c) Baseline Generation and Temporal Comparison

The “patient-specific conductivity baseline” provides a stable reference for longitudinal analysis. By comparing subsequent volumetric conductivity maps—acquired during or after therapy—against this baseline, the system can isolate transient or treatment-induced biological changes from the static background anatomy. This comparative approach facilitates the detection of subtle shifts in tissue state that might otherwise be obscured by anatomical noise or inter-patient variability.

A method according to some embodiments, further include translating, via an AI-based module, reconstructed electrical impedance tomography (EIT) images into synthetic anatomical representations by applying a trained image-to-image deep learning model to said reconstructed images.

A method exemplifying some embodiments of the invention, further includes storing temporal reconstructed volumetric conductivity maps; and identifying temporal trends in biological response by comparing conductivity values across said temporal reconstructed volumetric conductivity maps. In some embodiments at least one of the temporal reconstructed volumetric conductivity maps is reconstructed following ionizing radiation delivery; and the identifying of the temporal trends includes identifying conductivity changes induced by the ionizing radiation delivery.

The method according to some embodiments includes verifying, via the processing unit, a beam range of a particle therapy beam by identifying a localized increase in conductivity corresponding to a Bragg peak location.

According to some embodiments the method, further includes generating, via the processing unit, a clinical alert when a change in one or more volumetric conductivity map exceeds a predefined threshold.

In some embodiments the method includes exporting data derived from one or more volumetric conductivity map to a treatment planning system (TPS); and recalculating a radiation dose based on said exported data.

In some examples the method includes providing a patient support assembly having a tabletop where one or more portions of this tabletop is radiolucent and includes radiolucent electrodes.

The method may include integrating at least one internal electrode into a catheter needle or a brachytherapy applicator, and reconstructing the volumetric conductivity maps using electrical impedance measurements generated between the internal electrode and at least one of said plurality of radiolucent electrodes.

According to some aspects of the present invention a non-transitory computer-readable medium storing instructions that, when executed by a processing unit, cause the processing unit to perform a method for biologically-guided monitoring and treatment assistance. The method includes:
receiving a spatial orientation of a plurality of radiolucent electrodes relative to a target anatomical region of a patient from a surface detection module;
receiving impedance data from an impedance acquisition unit coupled to the radiolucent electrodes;
generating a spatial co-registration of said spatial orientation with anatomical imaging data of the target anatomical region to define a digital volumetric mesh;
reconstructing at least one volumetric conductivity map by mapping the received impedance data onto said volumetric mesh; and
superimposing one or more volumetric conductivity maps onto the anatomical imaging data to identify at least one biological response not characterized by the anatomical imaging data.

According to some aspects of the present invention, a radiolucent immobilization interface for use in a biologically-guided radiotherapy monitoring system, includes:
radiolucent substrate configured to conform to a target anatomical region of a patient and maintain said target anatomical region in a fixed position relative to a radiation delivery source;
radiolucent electrodes integrated into or disposed upon said substrate, wherein the electrodes are positioned to establish electrical contact with the skin of the patient when the substrate is in an immobilization position; and
radiolucent conductive leads coupled to the radiolucent electrodes, said leads extending to an interface connector configured for electrical coupling to an external impedance acquisition unit.

In some embodiments the radiolucent substrate includes a thermoplastic material configured to be molded to the patient's specific anatomical contours and to retain said contours upon cooling. In some cases one or more of the radiolucent electrodes is coupled to a radiolucent biasing element configured to maintain constant physical contact between said electrode and the patient's skin.

### BRIEF DESCRIPTION OF THE DRAWINGS

A complete understanding of the present embodiments and the advantages and features thereof will be more readily understood by reference to the following detailed description when considered in conjunction with the accompanying drawings. Embodiments of the present disclosure are illustrated as examples and are not limited by the figures of the accompanying drawings, in which like references may indicate similar elements and in which:

FIG. 1 illustrates schematically a system setup for biologically-guided adaptive radiation therapy during simulation, according to some embodiments of the invention.

FIG. 2 illustrates schematically a cross-sectional view of a thermoplastic immobilization mold and an integrated spring-loaded electrode assembly, according to some embodiments of the invention.

FIG. 3A illustrates schematically a system configuration for thoracic or abdominal radiation therapy including a thermoplastic mold fixated to a tabletop, according to some embodiments of the invention.

FIG. 3B is a perspective view of a contoured thermoplastic mold with integrated carbon-based electrodes and conductive wires, specifically configured for immobilization and electrical impedance tomography of the head and neck regions, in accordance with some embodiments.

FIG. 3C is a plan view of a planar thermoplastic mold integrated with an array of carbon-based electrodes and conductive wires, configured for subsequent molding to a patient's thorax, abdomen, or pelvis, in accordance with some embodiments.

FIG. 3D is a perspective view of a specialized treatment tabletop including integrated carbon-based electrodes and conductive wires, configured to provide posterior electrical interface in regions not covered by a thermoplastic mold, in accordance with some embodiments.

FIG. 4 illustrates schematically a cross-sectional anatomical view of a patient during particle therapy, showing the spatial relationship between irradiation beams and anticipated conductivity changes, according to some embodiments of the invention.

FIG. 5 is a schematic flowchart showing an overview of the primary stages for generating a patient-specific conductivity baseline and real-time EIT images, in accordance with some embodiments of the present invention.

FIG. 6 is a high-level system architecture diagram for a biologically-guided electrical impedance tomography system, in accordance with some embodiments.

### DETAILED DESCRIPTION OF EMBODIMENTS OF THE INVENTION

The following description provides illustrative, non-limiting embodiments of the present disclosure. It is to be understood that variations and modifications can be made without departing from the scope of the disclosure. Unless otherwise stated, all components described may be adapted or substituted with functionally equivalent alternatives.

As established in the background of the present disclosure, traditional EIT systems suffer from a high sensitivity to electrode positioning and a lack of spatial resolution that often renders the resulting images clinically insufficient for localized treatment monitoring. These challenges are particularly acute in the domain of radiation therapy, where existing imaging modalities (e.g., CT, MRI, CBCT) provide structural information but remain blind to real-time biological and ionization effects and functional tissue changes.

Furthermore, there is a need to provide a stable, reproducible interface that can function across multiple treatment fractions while minimizing image artifacts or interfering with the delivery of therapeutic radiation. Prior approaches have lacked a mechanism to co-register the low-resolution functional data of EIT with the high-resolution structural data of anatomical imaging, resulting in a “spatial gap” that prevents the precise localization of biological responses and temporal synchronization.

According to an aspect of the present disclosure, there is provided a biologically-guided monitoring and treatment assistance system. The system comprises a data interface configured to receive signals from radiolucent electrodes; a surface detection module configured to identify a spatial orientation of the radiolucent electrodes relative to a target anatomical region of a patient; an impedance acquisition unit coupled to the radiolucent electrodes; and a processing unit configured to:
(i) generate a spatial co-registration of said spatial orientation from the surface detection module with anatomical imaging data of the target anatomical region to define a digital volumetric mesh;
(ii) reconstruct one or more volumetric conductivity map from data provided by the impedance acquisition unit by mapping said data onto the volumetric mesh; and
(iii) superimpose said volumetric conductivity map onto the anatomical imaging data to identify one or more biological response not characterized by the anatomical imaging data.

According to some embodiments, the present disclosure provides for the integration of EIT data with various anatomical and functional imaging modalities. These may include, but are not limited to, computed tomography (CT), cone-beam computed tomography (CBCT), magnetic resonance imaging (MRI), positron emission tomography (PET), single-photon emission computed tomography (SPECT), fluoroscopy, X-ray imaging, and surface-guided systems. In these examples, such modalities are utilized to enhance the spatial resolution and accuracy of EIT reconstructions, guide electrode localization, and facilitate the co-registration of conductivity-based data with anatomical references to support clinical decision-making.

According to some embodiments, the present disclosure provides for the integration of EIT data with various anatomical and functional imaging modalities. These may include, but are not limited to, computed tomography (CT), cone-beam computed tomography (CBCT), magnetic resonance imaging (MRI), positron emission tomography (PET), single-photon emission computed tomography (SPECT), fluoroscopy, X-ray imaging, and surface-guided systems. In these examples, such modalities are utilized to enhance the spatial resolution and accuracy of EIT reconstructions, guide electrode localization, and facilitate the co-registration of conductivity-based data with anatomical references to support clinical decision-making.

In certain embodiments, specialized immobilization systems are employed that incorporate stable, non-artifact-producing electrode arrays. For example, patient-specific thermoplastic molds or treatment tabletops may be provided with embedded carbon-based electrodes and wire conductors. Such configurations are designed to enhance high-fidelity and reproducible impedance measurements while minimizing image artifacts and maintaining the integrity of the planned treatment dose across multiple sessions. According to some embodiments, these arrangements enable comprehensive volumetric coverage, including posterior regions, thereby improving the completeness and quality of the electrical impedance tomography reconstructions. Furthermore, some embodiments support closed-loop or adaptive workflows, providing dynamic biological feedback to assess treatment effectiveness and facilitate the optimization of therapeutic interventions in real time or across discrete treatment fractions.

According to further examples, the system includes thermoplastic immobilization molds embedded with carbon-based electrodes and conductive wires, configured for reproducible skin contact and compatibility with radiological imaging. A multichannel impedance acquisition subsystem may be used to collect voltage responses during or after a treatment session, which are subsequently processed by a computing workstation to generate volumetric conductivity maps. These maps may be co-registered with CT, MRI, CBCT, surface scans, or optical models to visualize treatment effects and biological responses. In some embodiments, the system supports adaptive treatment workflows, Bragg peak validation in particle therapy, and the monitoring of off-target tissue effects. Additionally, a baseline conductivity model—referred to in some examples as a “real EIT initial image” may be generated from anatomical segmentations to provide a reference for comparison. Modular configurations may also enable supine or upright treatment and brachytherapy, including the use of catheters or applicators configured to serve as electrodes for internal or intracavitary impedance acquisition.

As detailed in subsequent sections, the system in some embodiments supports a broad range of clinical use cases. These include the identification of treatment failure through the absence or mislocalization of conductivity changes, verification of SBRT precision, and Bragg peak range estimation in proton and heavy-ion therapies based on localized conductivity spikes. Certain examples also enable conductivity-based monitoring during brachytherapy and support both supine and upright radiotherapy workflows. Furthermore, pediatric applications, modular mold configurations, and retrofitting options may be utilized to expand the clinical utility and flexibility of the platform.

The specific details of the single embodiment or variety of embodiments described herein are to the described system and methods of use. Any specific details of the embodiments are used for demonstration purposes only, and no unnecessary limitations or inferences are to be understood thereon. Before describing in detail exemplary embodiments, it is noted that the embodiments reside primarily in combinations of components and procedures related to the system. Accordingly, components have been represented, where appropriate in the drawings, showing only those details that are pertinent to understanding the embodiments of the present disclosure so as not to obscure the disclosure with details that will be readily apparent to those of ordinary skill in the art having the benefit of the description herein.

The systems and methods described in the following embodiments integrate real-time electrical impedance tomography (EIT), anatomical imaging modalities, and stable electrode immobilization techniques to facilitate biologically-guided adaptive radiation therapy. These embodiments are applicable across a plurality of anatomical regions, radiation delivery modalities, and imaging workflows. For clarity, the present disclosure is described in detail with reference to its principal subsystems, clinical integration strategies, and exemplary embodiments.

According to some embodiments, the biologically-guided adaptive radiation therapy system comprises multiple integrated subsystems configured to enhance high-fidelity electrical impedance tomography (EIT) imaging during ionizing radiation treatments. These embodiments facilitate the acquisition, reconstruction, and analysis of real-time tissue conductivity data in conjunction with anatomical imaging modalities, providing a foundation for dynamic, biologically-informed treatment monitoring and adaptation.

### Hardware Subsystems and Patient Interfaces According to Some Embodiments:

### Thermoplastic Mold With Embedded Electrodes and Wires

A patient-specific immobilization device fabricated from a low-temperature thermoplastic material is configured to conform to a subject's anatomy. The mold incorporates electrodes embedded within or proximate to its inner surface for skin contact, with carbon-based conductive wires routed along the outer surface. The electrodes may be formed from graphite, carbon fiber, or similar carbon-based materials selected to minimize imaging artifacts, maintain dose distribution integrity during treatment planning and delivery, and reduce electromagnetic interference.

### Treatment Tabletop With Integrated Electrodes

A treatment tabletop comprising a substantially flat, carbon-fiber composite surface configured for placement atop a standard treatment bed. The tabletop includes embedded carbon-based electrodes and conductive wiring. These electrodes may be formed from graphite, carbon fiber, or similar materials and the wires may be composed of multiple strands of carbon fiber, selected for their low atomic number and compatibility with imaging modalities such as CT, CBCT, and MRI. The tabletop is configured to serve both as a mechanical platform for patient positioning and as an electrical interface for posterior or lateral EIT coverage. The tabletop may include extension interfaces configured to accommodate electrode inputs from the thermoplastic mold, thereby forming a unified electrode network with shared signal routing to an electronics box.

### Headrest With Electrode Integration

For head and neck or cranial applications, a specialized immobilization headrest may be employed. This component may include embedded carbon-based electrodes configured to enhance volumetric coverage while maintaining compatibility with MRI, CT, and radiation delivery protocols.

### Electronics Box

A signal generation and acquisition unit is configured to inject low-amplitude alternating current and measure resulting voltage differences across an electrode array. The electronics box may be implemented as a standalone external unit, such as on a mobile trolley, or integrated directly into a mechanical structure of a treatment or scanning bed. The unit is electrically connected to the embedded electrodes via multichannel carbon fiber cables and may include separate input terminals to distinguish between electrode arrays located in a tabletop and those embedded in a thermoplastic mold or headrest. This modular port configuration is intended to facilitate traceability, system calibration, and flexible electrode assignment across various clinical configurations.

### Interconnect System

Multichannel, shielded, carbon-based cables route signals between the electrodes and the electronics box. These may be detachable and color-or channel-coded to support traceability and reduce configuration error. The connectors from the mold interface directly with the electronics box, or indirectly via the tabletop extension interface. Workstation and Software Suite: A dedicated computing platform located outside the treatment room receives measurement data from the electronics box via Ethernet or, in alternative embodiments, a wireless connection (e.g., WiFi). Data is typically transmitted in . eit format or a comparable structured file format. The workstation is configured to execute a proprietary or open-source software suite that may optionally integrate open-source frameworks and toolkits. This software performs real-time EIT image reconstruction, co-registration with anatomical imaging, trend analysis of conductivity data, and visualization.

### Workstation and Software Suite

A dedicated computing platform located outside the treatment room receives measurement data from the electronics box via Ethernet or, alternatively a wireless connection (e.g., WiFi). Data may be transmitted in . eit format or a comparable structured file format. The workstation may run a proprietary or open-source software suite that may optionally integrate open-source frameworks and toolkits. This software performs real-time EIT image reconstruction, co-registration with anatomical imaging, trend analysis of conductivity data, and visualization.
In addition the software may include advanced analytical tools for identifying abnormal conductivity patterns, tracking temporal changes across treatment fractions, and correlating biological response with treatment outcome. It may also manage internal databases for patient-specific conductivity baselines, session metadata, reconstruction parameters, and system logs. The software may integrate clinical decision support modules, machine learning engines, or direct interfaces to oncology information systems (OIS), picture archiving and communication systems (PACS), or treatment planning systems (TPS).
Within the treatment room, a secondary screen may be used to display full or partial EIT reconstructions in real time, supporting both setup guidance for clinical staff and optional visual feedback mechanisms for patient engagement.
The software may support Bragg peak range estimation in proton and heavy-ion therapies. This is achieved by identifying localized conductivity spikes near the distal end of the ion track, where linear energy transfer (LET) and biological impact are maximized. The system may visualize these features in real time or flag deviations from expected patterns to assist in adaptive treatment workflows.

### Integration With Clinical Imaging and Treatment Systems

The subsystems and components are designed for compatibility with conventional radiotherapy infrastructure, including linear accelerators (LINACs), upright treatment chairs, and relevant immobilization accessories. Embedded imaging modalities (e.g., CBCT, MRI, PET, portal imaging) and external systems (e.g., on-rails CT, surface-guided imaging, thermal imaging, X-ray systems) may be used to provide anatomical priors or track anatomical changes. The system is adaptable to treatment workflows involving photon, proton, electron, or radionuclide-based therapies.

### Electrode and Boundary Identification Pipeline

Initial electrode positioning, body contours, and anatomical features are extracted from simulation CT or MRI scans. During treatment, supplemental imaging data from CBCT, X-ray, or surface imaging may be used to update or verify this information. Data processing may be performed via manual segmentation, rule-based algorithms, or AI-based methods.

### Treatment Configuration Flexibility and Modular Deployment

in scenarios where the use of a tabletop is not feasible (e.g., upright radiotherapy), electrode arrays may be distributed across multiple thermoplastic mold sections positioned anteriorly, posteriorly, or laterally relative to a subject. These embodiments may further include features such as multi-session reuse and adaptation for upright therapy. In certain examples, the system is configured to integrate dose-based or biology-based feedback to facilitate real-time or inter-fraction treatment adaptation.

### Thermoplastic Immobilization With Embedded Electrodes According to Some Embodiments

A patient-specific immobilization component may include one or more thermoplastic sheets embedded with carbon-based electrodes and conductive pathways. These molds conform anatomically to the patient's surface and serve a dual purpose: (1) to stabilize patient positioning during imaging and radiation treatment; and (2) to ensure high-fidelity, reproducible skin contact for electrical impedance tomography (EIT) signal acquisition.

### Materials and Composition Examples

Each thermoplastic mold is fabricated from a low-temperature, shape-retentive thermoplastic material such as polycaprolactone. The electrodes are composed of graphite, carbon fiber, or similar carbon-based electrically conductive materials chosen to avoid imaging artifacts, maintain radiation dose distribution integrity, and minimize electromagnetic interference. Conductive pathways are implemented using multi-strand carbon fiber wires.
The conductive wires are embedded within the body of the thermoplastic material itself, either:
   By sandwiching the wires between two thermoplastic layers during fabrication; or
   By co-extruding or laminating the thermoplastic and carbon-based wiring during mold manufacture, allowing seamless integration of the conductors; or
   Embedding the wires by melting and compressing the thermoplastic material around them during a controlled molding process, where the thermoplastic is softened, the wires positioned, and the material subsequently compressed and cooled to lock the wires in place.
Other equivalent manufacturing methods may also be employed, provided that the resulting structure securely incorporates the conductive elements within the thermoplastic body while preserving mechanical flexibility, anatomical conformity, and compatibility with radiation treatment and imaging protocols.
This wire-embedding technique is intended to reduce external routing and improve durability, while simplifying clinical use and maintaining a smooth patient-facing surface during positioning.

### Electrode-Skin Contact Strategies

For consistent and secure skin contact, spring-loaded electrodes may be incorporated into the mold. These electrodes feature:
   A compressible internal spring, made from non-conductive, low atomic number material (e.g., polyether ether ketone, PEEK), that pushes the conductive electrode element toward the skin.
   A central channel allowing passage of the carbon fiber wire through the spring body, maintaining electrical continuity.
The spring-loaded design compensates for minor surface irregularities or anatomical asymmetries, improving contact uniformity without requiring excessive mold pressure.

### Headrest and Modular Sections

For head and neck treatments, a modular headrest is employed. The headrest is constructed to accommodate various cranial shapes and sizes and includes embedded carbon-based electrodes and conductive pathways similar to those in the thermoplastic mold. Sections may be snapped or slid into adjustable support arms or baseplates for accurate, reproducible positioning.
In certain configurations (e.g., upright radiotherapy or seated nuclear medicine procedures), the standard mold-tabletop combination may be replaced with multiple interlocking thermoplastic pieces, anterior and posterior, each containing integrated electrodes. These pieces are designed to snap together via integrated clips or magnetic latches, forming a stable, full-coverage immobilization assembly.

### Fixation and Positioning

Thermoplastic molds and modular components may include frame-embedded clip systems that secure the immobilization device to the treatment tabletop, scanning couch, or upright treatment chair. This is to enhance rigid mechanical fixation, minimizes inter-fraction variation, and supports reproducible electrode positioning.

### Electrode Layout and Imaging Dependency

The embedded electrodes may be distributed uniformly or non-uniformly across the mold, depending on the clinical objective and anatomy of interest. However, once the mold is heated and shaped to the individual patient, electrode locations shift according to surface deformation. For this reason, the final electrode positioning and external patient contour must be extracted after molding, using one or more anatomical imaging modalities.
These modalities may include tomographic techniques such as CT, CBCT, or MRI, or non-tomographic systems such as surface-guided imaging, orthogonal X-rays, or planar radiographic localization systems. The extracted spatial coordinates are then used to define the boundary geometry, surface mesh, and electrode placement model required for accurate EIT reconstruction. Electrodes may vary in shape, depth, and diameter, with selection guided by treatment site, signal sensitivity requirements, and manufacturability. Fiducial markers or embedded identifiers may optionally be included to assist with electrode localization in imaging studies.

### Manufacturing Considerations

The thermoplastic molds may be pre-fabricated with integrated electrodes and wires to support streamlined clinical workflows and high-throughput deployment.
Manufacturing strategies may prioritize:
   Compatibility with standard clinical imaging workflows;
   Artifact-free performance in planning scans;
   Efficient setup and takedown for daily treatment sessions; and
   Modularity to accommodate diverse patient anatomies.

### Mold Variants and Catalogued Configurations

To accommodate different treatment sites, patient anatomies, and clinical protocols, the system may include a catalog of pre-configured thermoplastic mold variants. These molds differ in size, shape, electrode density, and geometrical distribution, and may be tailored to specific anatomical regions such as the head and neck, thorax, breast, abdomen, pelvis, or extremities.
Molds may be selected based on:
   Treatment modality (e.g., photon, proton, electron, radionuclide therapy),
   Patient size and positioning requirements (e.g., supine vs. upright),
   Electrode configuration (e.g., full 360° coverage vs. partial arc),
   Imaging compatibility constraints (e.g., MRI-safe vs. CT-optimized configurations).
   Patient demographic, including specially scaled designs for pediatric patients or individuals with atypical body dimensions.
Mold variants may include embedded identifiers (e.g., barcodes, alphanumeric markings, RFID) to support session traceability, software configuration, and electronic documentation. In some embodiments, custom molds are generated for complex cases using additive manufacturing, semi-automated templates, or clinician-guided design workflows.

### EIT Signal Generation and Measurement Subsystem

The electrical impedance tomography (EIT) subsystem is responsible for generating excitation signals, capturing resulting voltage responses, and converting raw impedance data into imaging-ready datasets. According to some embodiments, the system is configured to support high-resolution, real-time monitoring of radiation-induced changes in tissue conductivity, with a focus on volumetric (3D) reconstructions within clinical contexts.

### Electrode Channel Architecture

To enable three-dimensional (3D) image acquisition, the system may employ a large number of electrodes organized into multiple circumferential arrays (e.g., electrode rings) positioned at different anatomical heights. Some configurations utilize 128 to 256 electrodes or more, depending on the anatomical region, patient size, clinical application, or the technological evolution of the imaging system. Higher electrode counts support improved spatial resolution, greater volumetric coverage, and enhanced reconstruction fidelity in both superficial and deep tissues.
The electrodes may be addressed using multiplexed switching matrices capable of dynamically selecting electrode pairs for current injection and voltage measurement. The system may support various drive patterns including adjacent injection, skip-one, skip-two, or custom configurations based on anatomical requirements. The voltage measurements may be acquired in parallel and include both real and imaginary components, forming complex impedance values for each electrode pair. These complex values reflect the in-phase (resistive) and out-of-phase (reactive) electrical properties of tissues, enabling the derivation of frequency-dependent conductivity and permittivity maps.
The real voltage component corresponds to resistive (in-phase) responses of tissues, while the imaginary component captures capacitive or reactive (out-of-phase) behaviors. These components are essential for accurate modeling of tissue impedance and biological response under alternating current stimulation.
To support complete anatomical coverage, the electrodes may be distributed across different structural components of the system:
   In some configurations, a subset of electrodes is embedded in the thermoplastic immobilization mold, which conforms anatomically to the anterior or lateral surfaces of the patient.
   A complementary subset of electrodes may be embedded in the carbon fiber tabletop and/or headrest, providing coverage of posterior regions that cannot be reached by the anterior mold alone.
   In alternative configurations—such as those used in upright treatments, or for increased flexibility—two or more thermoplastic mold sheets may be employed, one for the anterior side and one or more for posterior or lateral sides, each pre-integrated with carbon-based electrodes and conductive wiring. These molds interlock or clip into position to provide full circumferential and longitudinal electrode coverage.
This modular distribution enhances:
   Volumetric EIT coverage across front, back, and lateral surfaces;
   Stable and reproducible electrode contact across fractions, enabled by fixed positioning within rigid or semi-rigid immobilization structures;
   Flexible deployment across supine, prone, or upright treatment configurations. Electrode layout and array geometry are coordinated across all structural components to maintain consistent inter-electrode spacing and signal integrity throughout the 3D acquisition process.

### Signal Generation and Acquisition

The electronics subsystem includes precision signal generators configured to inject low-amplitude alternating current (AC) through selected electrode pairs and simultaneously measure the resulting voltage responses across other electrode combinations. The injected currents and measured voltages are used to compute complex impedance values (real and imaginary components) which serve as the basis for reconstructing tissue conductivity distributions.
The system may support single-frequency, multi-frequency excitation protocols, as and single-current, multi-current injection schemes, and combinations thereof. These protocols may be selected manually or configured automatically based on the anatomical site, patient characteristics, treatment modality, or desired imaging resolution.
   In single-frequency mode, a fixed frequency (e.g., 100 kHz) is used for all current injections, optimizing temporal resolution and computational simplicity.
   In multi-frequency mode, the system sequentially or simultaneously applies current injections at multiple frequencies (e.g., ranging from tens of kilohertz to several megahertz), enabling the characterization of frequency-dependent conductivity and permittivity across different tissue types.
   In single-current mode, a fixed current amplitude is used (e.g., 5 mA RMS), consistent with established medical safety thresholds.
   In multi-current mode, the system varies the amplitude of the injected currents either across electrodes or over time to enhance sensitivity or to explore the nonlinear electrical behavior of irradiated tissues.
These flexible signal injection protocols allow:
   Adaption to tissue-specific electrical properties
   Improving signal-to-noise ratios under varying impedance conditions
   Capturing dispersive behavior of tissues to infer structural or biochemical changes
   Supporting advanced reconstruction algorithms that incorporate spectral data
   Enabling dual-or tri-modal imaging when fused with anatomical datasets from CT, CBCT, or MRI
Voltage sensing pathways typically include differential low-noise amplifiers and are digitized via high-speed, high-resolution analog-to-digital converters (ADCs). Sampling and multiplexing are typically synchronized with current injection to ensure phase coherence and prevent signal contamination from switching artifacts or motion-induced delays.
In some embodiments, the system may also support:
   Modulated or frequency-swept injections (e.g., chirp signals),
   Waveform shaping for tissue-specific impedance profiling,
   Gated or time-resolved acquisition protocols synchronized with physiological events (e.g., respiratory or cardiac cycles),
   And artifact rejection routines to mitigate motion or external electromagnetic interference.
Injection parameters are constrained within medically accepted limits for electrical safety in human subjects and may be governed by configurable software safety layers and hardware interlocks.

### Data Interface and Integration

Measurement data acquired by the electronics subsystem—including real and imaginary voltage components for each stimulated electrode pair—is transmitted to a dedicated workstation that in some cases is located outside the treatment room. Communication may be established via a wired Ethernet interface for high-speed, low-latency data transfer. In alternative embodiments, wireless communication protocols, such as Wi-Fi or short-range optical links, may be employed to reduce cabling and simplify integration with mobile or modular setups.
The data may be transferred in a structured format (e.g., . eit or an equivalent extensible file format), containing relevant metadata including:
   Electrode stimulation and measurement configurations,
   Injection parameters (current amplitude, frequency, waveform),
   Time stamps, and
   Session identifiers.
The workstation may run a proprietary or open-source software suite, optionally extended with open-source libraries, which performs real-time EIT image reconstruction and analysis. The software includes modules for:
   Complex signal preprocessing and noise filtering,
   Finite element model-based image reconstruction,
   Trend analysis across multiple treatment sessions,
   Data export to PACS, TPS, or OIS systems, and
   Visualization of conductivity maps overlaid on anatomical models or imaging scans.
In addition to the primary imaging modalities used for internal anatomical localization (e.g., CT, CBCT, MRI), the system may also integrate external surface scanning methods to refine body contours and electrode positioning. In certain configurations, the system employs optical cameras—including structured light scanners, stereo photogrammetry, or RGB-D sensors—to acquire 3D surface models of the patient's external anatomy and electrode locations. These surface models can be:
   Used as standalone boundary meshes for EIT reconstruction,
   Co-registered with internal imaging for enhanced model accuracy, or
   Compared across fractions for setup reproducibility analysis.
This additional modality may allow for non-ionizing, fast, and operator-independent acquisition of external geometry, which is particularly advantageous in pediatric cases, upright radiotherapy, or when repeated CT/CBCT scans are undesirable or unavailable.
Electrode identification on the body surface may be automated using:
   Optical markers or fiducials on electrode housings,
   Color-coded features or reflectivity-based segmentation,
   Machine vision algorithms integrated into the software pipeline.
Combined with internal anatomical priors, this surface data may enhance the fidelity of the forward model, reducing localization errors and improving the accuracy of conductivity reconstructions.
The data and metadata may be stored in a structured session log, allowing for reproducibility, version tracking, and retrospective analysis of treatment response across multiple fractions or imaging sessions.

### Software and Data Processing

According to some embodiments, a proprietary or open-source software platform is configured to facilitate data acquisition, real-time image reconstruction, anatomical data integration, and temporal tracking of physiological or ionization-induced changes. The software may operate on a dedicated workstation—which in certain examples is located outside the treatment room—and is modularly designed to support algorithmic enhancement, clinical usability, and data integrity.

### Image Reconstruction Algorithms

In some exemplary embodiments, the initial image reconstruction engine is based on an adaptation of the EIDORS framework, an open-source toolkit for electrical impedance tomography (EIT). According to some embodiments, the reconstruction logic is implemented within a proprietary or open-source Python-based environment. The system is configured to support custom, proprietary or open-source reconstruction algorithms optimized for high-performance and high-accuracy volumetric imaging. The system may support:
   2D reconstruction, for slice-based visualization in planar geometries;
   Quasi-3D reconstruction, via stacked or interpolated 2D slices; and
   True 3D volumetric reconstruction, using finite-element models (FEM) and/or anatomical priors from CT/MRI.
Reconstruction is performed in real-time, with a minimum target of 5 frames per second, enabling biological changes to be visualized dynamically during radiation delivery. Algorithmic methods may include but are not limited to:
   Gauss-newton iterative solvers,
   Gradient-based regularized inversion,
   Greit-based reconstruction pipelines, and

Custom FEM solvers tailored to patient-specific meshes.
Additional versions may include AI-assisted or data-driven EIT solvers designed to improve speed and resolution under noisy or ill-posed conditions.

### Enhancement

To improve spatial resolution and anatomical accuracy, the reconstruction pipeline may incorporate multiple enhancement methods, including:
   Anatomical priors obtained from CT, MRI, or CBCT scans. These priors include segmented tissue regions and organ boundaries, which are used to inform conductivity regularization, mesh adaptation, and reconstruction constraints.
   Boundary modeling and external contouring, using data from cone-beam CT (CBCT), surface imaging systems, or optical scanning technologies (e.g., structured light, stereo depth cameras, photogrammetry).
   AI-based image-to-image translation, trained to convert raw EIT conductivity maps into synthetic anatomical representations in the CT, CBCT, or MRI domain. These networks may include convolutional neural networks (CNNs), generative adversarial networks (GANs), transformers, or other deep learning architectures, trained using proprietary or open source datasets. Inference may be conducted locally within the hospital's secure environment, or remotely via encrypted connection to cloud servers, depending on clinical preferences and information security policies. These enhancement methods and the associated reconstruction architecture are configured to evolve with technology and data availability, allowing for the integration of higher-performing models or novel imaging-guided reconstruction techniques.

### Co-Registration and Co-Localization

Accurate image interpretation requires precise spatial co-registration between EIT reconstructions and anatomical datasets. The system may provide both rigid and deformable co-registration algorithms, including:
   Standard affine or B-spline methods,
   Landmark-based alignment using fiducials or anatomical features,
   AI-guided co-registration leveraging learned correspondences from paired imaging data. Electrode localization, external contouring, and anatomical segmentation may be performed via semi-automatic pipelines, where results from automated tools may be presented to the user for optional confirmation and editing. This may apply to:
   Initial localization during CT/MRI simulation, and
   Per-fraction updates using CBCT, optical scans, or other available imaging modalities.
Data may be co-registered in a unified coordinate space to allow overlay and fusion between EIT data, dose maps, anatomical images, and temporal information.

### Temporal Tracking and Longitudinal Analysis

Reconstructed conductivity maps may be time stamped and stored to allow longitudinal analysis across treatment fractions. The software may track changes in conductivity within defined regions of interest (ROIs), such as tumor volumes or organs-at-risk, enabling clinicians to:
   Identify deviations from baseline conductivity patterns
   Correlate observed changes with dose delivery or anatomical motion
   Quantify potential treatment response or toxicity biomarkers over time
The software may be extensible to support time-aligned datasets, including future integration with treatment delivery logs, physiological signals (e.g., ECG, respiratory gating), and radiation dosimetry records. These features may enable biologically adaptive re-planning in future versions of the system.

### Anatomically-Informed Forward Modeling and Baseline Conductivity Mapping:

In some examples, to enhance the fidelity, robustness, and clinical interpretability of EIT reconstructions, the present invention introduces a novel initialization method, referred to as the “real EIT initial image”. This anatomically-informed approach uses segmented imaging data and organ-specific conductivity references to generate a realistic, patient-specific conductivity map prior to EIT image acquisition. This map serves both as a forward model solution and a baseline reference for biological tracking.
   Generation of the Real EIT Initial Image According to some Embodiments
The process includes the following steps:

### 1. Anatomical Segmentation

High-resolution anatomical imaging (CT, CBCT, or MRI) is acquired during simulation.
Automated, AI-assisted, or manual segmentation is applied to identify organs and tissue structures, including tumor, lungs, muscle, fat, and bone.

### 2. HU-to-Density Mapping

For CT/CBCT, each voxels Hounsfield Unit (HU) is converted into a relative mass density or electron density using calibration curves established during CT commissioning in radiotherapy departments.
MRI datasets may use alternative tissue classification features or synthetic mapping techniques.

### 3. Conductivity Assignment From Organ Tables

Segmented organs are matched to predefined organ-specific conductivity values using published tables and biophysical literature.
Each voxel is assigned a conductivity value based on its anatomical classification and density, generating a 3D conductivity distribution consistent with known human physiology.

### 4. Construction of the Real Eit Initial Image

The resulting map is used as an initial solution to the forward problem in EIT.
It may be used as a prior in iterative solvers, a constraint in inverse models, or a validation reference during real-time image updates.

### Use as a Baseline for Tumor and Healthy Tissue Monitoring According to Some Embodiments

The real EIT initial image may also serves as a baseline for distinguishing radiation-induced conductivity changes in both tumor and normal tissue regions to support:
   Quantitative tracking of biological response across treatment fractions,
   Early identification of tumor response or tissue damage,
   Longitudinal comparison of conductivity patterns using aligned session data,
   Predictive modeling of treatment outcomes, by comparing current measurements against this baseline.
This dual use—as a prior for reconstruction and a reference for biological change detection—enables biologically-guided adaptive radiation therapy based on tissue conductivity dynamics.

### Robustness and Uncertainty Handling

To improve real-world applicability, the system may integrate compensation mechanisms for:
   Variability in CT image quality, beam hardening, or misregistration errors;
   Inter-fraction motion, deformation, or posture changes, addressed via rigid or deformable image co-registration;
   Electrode-skin contact impedance, estimated from EIT data and/or anatomical features (e.g., subcutaneous tissue thickness), and incorporated into the boundary modeling of the forward problem.
In certain embodiments, the contact impedance may be visualized independently, allowing clinicians to identify poor electrode coupling or setup variability before or during treatment.

### Clinical Workflow Integration

The integration of the Electrical Impedance Tomography (EIT) system into the radiotherapy workflow may enhance treatment precision and enable real-time monitoring of tissue conductivity changes. This section outlines the integration process across various stages of patient care according to some embodiments.

### Simulation Session

Thermoplastic Mold Creation: A thermoplastic mold embedded with electrodes and carbon-based conductive wires is fabricated during the CT simulation session. The mold is heated to become pliable, carefully shaped to conform to the patient's treatment region (e.g., head, neck, thorax), and then cooled to harden, forming a rigid, anatomically matched immobilization device. This ensures reproducible patient positioning throughout the course of therapy. As part of this process, the radiation therapist or designated clinical operator must ensure:
   That each electrode makes full and stable contact with the patient's skin, without air gaps or deformation,
   That the embedded wires are properly routed to avoid mechanical stress or disconnection during mold placement or patient movement, and
   That each electrode connection is securely linked to the electronic EIT acquisition system, either directly or via interface cables.
Proper electrodes contact and connectivity are critical for achieving reliable and artifact-free impedance measurements, particularly during multi-fraction treatment protocols. Verification protocols (e.g., impedance checks or signal quality validation) may be incorporated into the software workflow to assist the clinical team.

### CT Acquisition

With the thermoplastic mold in place, a CT scan is performed to capture high-resolution anatomical information of the treatment region. This scan serves as the foundational dataset for both radiation therapy planning and EIT image reconstruction.

### CT-to-EIT Integration and Preprocessing

Following acquisition, the CT image dataset is imported into the proprietary EIT software platform. This may be performed either:
   Manually, by exporting the CT files from the Oncology Information System (OIS), Picture Archiving and Communication System (PACS), or local storage, or
   Automatically or semi-automatically, with optional approval or confirmation from clinical staff, depending on system configuration.
During typical clinical workflows, patients remain on the CT simulator bed for approximately 15-20 minutes after the scan to allow the thermoplastic mold to fully harden and set. This post-scan period is utilized by the EIT system to:
   Import and process the CT data,
   Extract the electrode positions, body contour, and anatomical segmentation required for EIT modeling, and
   Initialize the reconstruction framework using either anatomical priors or the Real EIT Initial Image model described above.

### EIT Baseline Acquisition

Once CT-derived parameters are available, the first EIT acquisition is performed prior to patient repositioning, using the newly immobilized setup. This measurement forms the baseline conductivity map, capturing tissue state prior to radiation therapy and serving as a reference for longitudinal comparison throughout the treatment course.
The system immediately begins real-time reconstruction of EIT images, providing clinicians with immediate feedback on conductivity distribution, signal quality, and electrode contact integrity.
In certain cases, MRI may be utilized as an alternative or supplementary imaging modality to establish the baseline, depending on clinical requirements and equipment availability.

### Daily Treatment Sessions in Some Embodiments of the Invention

### Mold Reapplication

At the beginning of each treatment session, the previously fabricated thermoplastic mold with embedded electrodes and wires is re-applied to the patient. This mold ensures consistent patient positioning and reproducibility of electrode contact across all fractions. The mold is clipped or fixed to the treatment couch or tabletop, matching the immobilization standard used in conventional radiotherapy masks.

### System Connection and Initialization

Once the mold is secured, the electrodes are connected to the electronic interface unit, and the EIT acquisition software is initialized. Signal quality checks and impedance validation may be performed automatically or under operator supervision. Real-time
EIT image generation begins at this point.

### Patient Positioning and Optional Imaging Import

For accurate positioning, standard radiotherapy imaging techniques such as cone-beam CT (CBCT), surface-guided radiation therapy (SGRT), orthogonal kV X-rays, or others may be used. Optionally—or when clinically required—the resulting images can be imported into the EIT software. This supplemental data may be used to:
   Update the anatomical model or external boundary shape,
   Refine electrode localization and/or contact impedance,
   Adjust conductivity priors or mesh geometry used in the reconstruction algorithm.

### EIT Imaging

EIT image acquisition begins prior to beam-on, providing pre-treatment conductivity measurements, and continues throughout radiation delivery. The system captures real-time conductivity changes during the irradiation period and monitors potential early biological responses.

### Treatment Delivery

With the patient immobilized and EIT imaging active, radiation is delivered according to the treatment plan. EIT monitoring continues uninterrupted during beam-on.

### Post-treatment EIT Acquisition and Disconnection

Following beam-off, the system continues to acquire EIT data for a designated post-irradiation window, capturing delayed conductivity effects. This acquisition typically lasts until the medical staff re-enters the treatment room, and may be extended by a few additional minutes if deemed clinically valuable.
Once data acquisition is complete:
   The electrodes are disconnected from the electronic station,
   The thermoplastic mold is removed from the patient,
   And the mold is stored for reuse in subsequent treatment fractions, ensuring session-to-session consistency in both positioning and measurement fidelity.

### Data Visualization

Throughout the session, EIT data is visualized in real time on a secondary display, enabling clinicians to:
   Confirm proper electrode contact and system functionality,
   Observe immediate physiological responses during and after treatment,
   And verify the quality and consistency of acquired signals.

### Data Flow Across Treatment Fractions in Some Embodiments

The system is designed to support consistent, multi-session acquisition and analysis of electrical impedance data throughout the entire course of radiation therapy. Its architecture enables:
   Tracking Conductivity Trends: EIT data from each treatment fraction is recorded, time-stamped, and aligned with prior sessions. This enables clinicians to monitor changes in tissue conductivity over time, which may serve as an early indicator of treatment response, edema, necrosis, or other biological effects. Such trends may inform adaptive therapy strategies, including dose adjustment, re-planning, or treatment intensification.
   Facilitating Longitudinal Analysis: The system maintains a secure, structured archive of conductivity maps, patient-specific anatomical priors, and treatment metadata. Clinicians and researchers can retrospectively analyze these datasets to quantify temporal evolution of conductivity patterns, correlate with dosimetric parameters, and investigate biological variability across patient populations.
   Supporting Future Enhancements: While current implementations focus on data acquisition and real-time visualization, future software releases may incorporate:
   Automated trend detection algorithms using statistical or AI-based methods,
   Threshold-based alerts for significant deviations from expected patterns,
   Integration with oncology information systems (OIS) for clinical decision support and treatment adaptation.

### Real-Time Vs. Offline Usage

The system according to some embodiments supports both real-time intra-treatment monitoring and offline post-treatment analysis, offering flexibility to meet diverse clinical and research needs.

### Real-Time Monitoring

In some examples the system according to the invention produces volumetric, real-time internal images based on measured electrical signals acquired from the patient's body. In some examples the images are generated at a minimum rate of 5 frames per second and visualized live during treatment. This capability may support:
   On-the-fly assessment of tissue conductivity changes
   Immediate verification of electrode contact and system integrity
   Dynamic monitoring of physiological responses to ionizing radiation.

### Offline Analysis

Following each session, the complete dataset—including raw voltage measurements, reconstructed conductivity maps, and metadata—is stored for offline reprocessing.
This enables:
   Quality assurance and signal integrity review,
   Research analysis using advanced or experimental reconstruction algorithms,
   Clinical reporting, documentation, or compliance with audit requirements.
Beyond conductivity monitoring, the system's real-time volumetric imaging capability may support additional radiation therapy applications, including:
   Internal motion tracking (e.g., tumor displacement, organ drift),
   Gating and synchronization with respiratory or cardiac cycles,
   Bioelectrical signal monitoring, such as electrocardiography (ECG) or electroencephalography (EEG),
   Functional imaging, potentially assessing perfusion, ion transport, or tissue integrity in real time.
These capabilities may provide broad clinical utility across the radiation therapy workflow.

### Adaptive Therapy and Decision Support Features

The system according to some embodiments is designed to support biologically adaptive radiation therapy, wherein real-time changes in tissue conductivity are interpreted as surrogate markers of biological response. These conductivity changes—detected during or after irradiation—are used to guide clinical evaluation and may, over time, inform treatment plan modification through integration with the treatment planning system (TPS).

### Interpretation of Conductivity Changes According to Some Embodiments

The interpretation of conductivity patterns is initially clinician-driven or clinician-supervised, supported by quantitative data analysis and semi-automated decision-support tools. Typical use cases may include:
   Verifying accurate targeting of the tumor, especially in scenarios with steep dose gradients or sensitivity to anatomical shifts, such as proton and heavy ion therapy;
   Confirming that off-target regions, particularly organs-at-risk (OARs), remain electrically stable, indicating minimal physiological disruption;
   Identifying potential early biological indicators of under-response (e.g., lack of change in tumor conductivity) or over-response (e.g., excessive change in normal tissue).
As additional clinical data are accumulated, statistical modeling and machine learning tools may be used to define minimum or optimal conductivity changes associated with effective tumor control, and thresholds for undesirable changes in critical structures. Quantitative detection of significant changes may include:
   Relative percentage change within defined ROIs (regions of interest),
   Spatial pattern deviation metrics, and
   Statistical outlier detection compared to previous fractions or population baselines.
Clinicians may be able to define custom anatomical or functional ROIs, including tumor sub regions, margin expansions, or sensitive anatomical landmarks, for conductivity tracking and report generation.

### Threshold-Based Alerts

According to some embodiments the system supports threshold-based alerting mechanisms, which may be activated when observed conductivity changes exceed user-defined critical levels (e.g., Δσ>X% or Y S/m). Alerts may include:
   On-screen notifications with color-coded severity indicators,
   Auto-generated session reports detailing the location, magnitude, and statistical significance of the observed change,
   PACS or OIS annotations, enabling retrospective review or triggering institutional QA workflows.
In some embodiments, the system is configured to provide auditory alerts, which may be customized or adjusted based on clinical requirements or user-defined parameters.

### Integration With Treatment Planning Systems (TPS)

According to some embodiments, the system is configured to operate as a standalone adjunct system, providing biological insight without requiring modification to a standard TPS workflow. Alternatively, the system may be interfaced or integrated with commercial TPS platforms through established medical imaging and data interchange protocols (e.g., DICOM RT, HL7, or FHIR).
Integration goals may include:
   Importing EIT data into the TPS via PACS or OIS for dose review, adaptive planning, or cumulative analysis;
   Generating annotated maps of tissue conductivity changes, with ROI-specific suggestions for re-evaluation;
   Flagging patients for re-planning based on clinician-defined thresholds or automated detection models;
   Facilitating TPS-driven plan updates, with final decision authority retained by the attending physician.
In some embodiments the adaptive recalculations are performed by the TPS.
However, in some embodiments the system software may propose dose modulation targets (e.g., suggested boost or sparing zones) to the TPS based on biological response indicators.

### Biological Outcome Modeling and AI-Driven Scenarios

In some embodiments the system incorporates AI-based outcome modeling, correlating conductivity trends with:
   Clinical outcomes, such as local control rates, progression-free survival, or treatment-related toxicity;
   Functional imaging biomarkers, including PET SUV metrics, diffusion-weighted MRI values, or perfusion data;
   Historical patient datasets, enabling predictive modeling of treatment response and guiding adaptive decision-making.
In some embodiments, AI-driven modules are configured to:
   Generate treatment adaptation recommendations based on observed physiological or conductivity response patterns;
   Calculate predictive indices for biological effectiveness or failure risk during an early course of therapy; and
   Identify biologically relevant sub-regions for dose redistribution, such as intra-tumoral boosts or enhanced protection of organs-at-risk (OARs).
Inference may be performed locally or via secure cloud-based services depending on institutional policy and regulatory requirements.

### Optional and Alternative Embodiments

The system according to embodiments of the invention is designed for modularity and adaptability, enabling clinical deployment across a wide range of treatment scenarios and facility infrastructures. The following embodiments represent optional configurations, fallback modes, or site-specific adaptations that may be implemented without departing from the scope of the invention.

### Expanded Anatomical Coverage

While head and neck applications are common clinical use cases, the system according to some embodiments supports thermoplastic molds designed for the thorax, breast, abdomen, pelvis, and extremities. The molds may be fabricated from carbon-electrode embedding process as described above, with variation in the electrode array geometry, density, and distribution tailored to the anatomy of interest. Pediatric-specific configurations and mold sizes may also be offered.

### Real-Time EIT Mode Without Anatomical Fusion

The system may be operated in a standalone EIT mode, without anatomical co-registration from CT, CBCT, or MRI. In this configuration, electrode positions and external contours are extracted from surface imaging modalities such as optical scanning or CBCT topograms. This mode allows full real-time 3D conductivity imaging even in settings where anatomical imaging is unavailable or undesirable. Predefined geometries are supported only in calibration or QA tools, not for patient imaging.

### Particle Therapy Compatibility

The system may be fully compatible with proton, electron, and heavy-ion therapies, with no need for fundamental hardware changes. For use in MRI-guided or magnetically sensitive environments, special connectors and low-interference modifications to the electronics box may be applied to maintain MR-compatibility.
In proton and ion therapy contexts, the system may support:
   Verification of beam alignment and internal targeting, and
   Quantification of conductivity changes along the path of particle deposition, enabling range tracking or Bragg peak validation.

### Flexible Temporal Acquisition Modes

The system nay support customizable imaging schedules, including:
   Pre-defined acquisition windows, such as pre-irradiation, beam-on, and post-treatment intervals;
   Triggered acquisition, based on respiratory cycles, ECG signals, or user-defined markers (future option).
While continuous imaging may be employed as a default operational mode, these alternative acquisition modes are configured to support additional functions, such as motion-gated acquisition or low-data-rate monitoring for longitudinal or extended studies.

### Modular Retrofitting of Existing Systems

In some embodiments, modular electrode kits are provided for integration into existing immobilization devices or treatment couches. These retrofit kits may include:
   Flexible or semi-rigid electrode strips that attach to standard thermoplastic masks,
   Adapter modules to connect external electrodes to the system's electronics box,
   Optional tabletop overlays or clip-on panels embedding posterior electrodes for enhanced coverage.

### Use Cases and Clinical Scenarios According to Some Embodiments

The system may support a variety of clinical applications in radiation oncology. By enabling real-time, volumetric, and biologically-informed electrical conductivity monitoring, it may provide clinicians with insight into tissue response during and after radiation delivery. The following use cases exemplify its clinical utility and potential impact on patient management.

### Early Treatment Failure Detection

The system may support early identification of suboptimal therapeutic effect by detecting:
   Absence or insufficiency of conductivity changes within the tumor volume across early fractions, potentially indicating poor biological response;
   Unexpected conductivity changes in off-target regions, including sensitive areas such as the brainstem, spinal cord, or other organs-at-risk (OARs);
   Geometric displacement or misalignment, where observed conductivity patterns do not match the expected irradiated volume.
In its initial deployment, the system may act as a biological imaging adjunct, providing reconstructed conductivity maps for clinician interpretation. Over time, with sufficient dataset collection and integration with anatomical and dosimetric systems, these biological patterns may inform automated risk scoring, early adaptation, or treatment interruption protocols.

### Precision Assessment in SBRT and Conventional Radiotherapy

In some embodiments the system enables daily verification of dose delivery geometry by comparing:
   The spatial location of conductivity changes captured by EIT, and
   The planned dose distribution from the treatment planning system (TPS).
This comparison can confirm that high-dose regions align with the targeted tumor volumes, and that adjacent tissues remain electrically—and biologically—unaffected.
While this is particularly valuable in stereotactic body radiation therapy (SBRT), which uses few high-dose fractions, it is equally applicable to conventional multi-fraction regimens, where deviations can accumulate over time. EIT data is acquired in every fraction, ensuring continuous quality assurance and positioning verification throughout the full course of therapy.
   Monitoring of Organs at Risk (oars)
The system can be configured to monitor specific organs-at-risk, such as the optic chiasm, spinal cord, bowel, lung, or heart, for signs of acute or cumulative tissue stress.
These regions may be:
   Predefined by treatment protocol, or
   Custom-defined per patient, based on anatomical anomalies, preexisting conditions, or clinician preference.
Thresholds for concern may be user-defined or set via population statistics, and in some versions, EIT trends are correlated with TPS-derived dose maps, enabling more refined decision support tools.

### Proton and Heavy-Ion Therapy: Bragg Peak Validation

In some embodiments the system offers unique capabilities in proton and heavy-ion therapy, where precise dose localization and range accuracy are critical to treatment success. While the system does not directly image the Bragg peak curve, it detects radiation-induced conductivity changes along the beam path, particularly in regions where ionizing interactions cause physiological and structural alterations.
Notably, at the Bragg peak, the Linear Energy Transfer (LET) of protons and heavier ions increases sharply, resulting in:
   Higher biological effectiveness compared to entrance or plateau regions of the beam,
   Greater disruption of cellular membranes, ionic environments, and tissue architecture,
   And consequently, larger and more localized conductivity changes in the irradiated tissue.
The system may be configured to detect these differential conductivity responses, with the drop-off in measured conductivity change correlates spatially with the end of the particle track, i.e., the Bragg peak. This phenomenon is dose-and tissue-dependent, and may manifest:
   Immediately, during or following the first fraction in high-dose regimens; or
   Progressively, requiring several fractions to become distinct in lower-dose protocols. These observations may enable:
   Verification of beam range, particularly in anatomically variable sites,
   Detection of range shifts or anatomical changes that affect dose distribution, and
   Development of LET-informed adaptive strategies, particularly in high-precision or re-irradiation contexts.

### Brachytherapy and Other Interventional Treatments

In some examples the system is applicable to brachytherapy and other interventional or implant-based radiation therapies, including:
   High-dose-rate (HDR) and low-dose-rate (LDR) brachytherapy,
   Interstitial and intracavitary applicator-based delivery.
In these treatment modalities, the system may be configured to:
   Verify applicator placement and orientation within the target anatomy,
   Track dynamic conductivity changes in tissues surrounding the implant or source dwell path,
   Assess local biological effects immediately before, during, and after dose delivery.
In certain embodiments, the brachytherapy applicator and/or the interventional catheter (as previously described) may themselves be used as one or more electrodes. In some cases, the applicator or catheter may be made of or coated with suitable electrically conductive, biocompatible, and imaging-compatible materials. These integrated-electrode applicators and interventional catheters may be used for:
   Direct tissue interface measurements at the source location,
   Enhanced spatial resolution in anatomically constrained areas (e.g., cervix, rectum),
   Or combined bipolar configurations (e.g., one electrode on the applicator, one on the surrounding mold or cavity wall).
Alternatively, when the applicator is not compatible or desired as an electrode, adjacent or surrounding thermoplastic molds with embedded electrodes may be used to acquire volumetric conductivity data in parallel.
This approach may enable:
   Real-time biological validation of source placement and expected dose deposition,
   Monitoring of tissue response asymmetries or unintended impact on surrounding organs, and
   Biologically adaptive dwell-time modulation, based on conductivity-derived response indicators.
As with external beam scenarios, this system may operate in standalone or PACS-integrated workflows, storing session data and visualizations for retrospective review, clinician interpretation, or integration with treatment planning systems.
   Tumor Necrosis vs. Inflammation Differentiation
The system according to embodiment of the present invention supports the differentiation between necrotic tissue and inflammatory response, based on:
   The direction, magnitude, and temporal evolution of conductivity changes,
   Or in future, through correlation with functional imaging or histopathological data.
This capability may guide timing of follow-up imaging, biopsy, or modification of supportive therapies, especially in cases where distinguishing tumor progression from pseudoprogression is clinically challenging.

### Technical Overview and Clinical Utility

According to some embodiments, the present invention relates to a biologically-guided adaptive radiation therapy system and method configured to integrate real-time Electrical Impedance Tomography (EIT) with anatomical imaging modalities and immobilization structures. By embedding carbon-based electrodes and conductive pathways into thermoplastic molds, tabletops, or headrests, the system may facilitate reproducible volumetric conductivity imaging during or after radiation delivery to allow:
   Monitoring of tissue response during treatment, which in some cases may enable real time biological feedback,
   Integration with existing radiotherapy protocols, including CT/MRI simulation, CBCT verification, and PACS/OIS interoperability,
   Implementation of a Real EIT Initial Image methodology, derived from CT/MRI segmentations and organ-specific conductivity tables for anatomically informed forward modeling,
   High-resolution, volumetric image reconstruction, with support for 2D, quasi-3D, and 3D EIT imaging modes,
   Configuration across external beam radiation, (e.g., proton, heavy ion, electron, photon), brachytherapy, target radionuclide therapy and intraoperative radiotherapy, with flexible mold geometries and electrode arrangements,
   Compatibility with multi-fraction workflows, maintaining baseline reproducibility for longitudinal conductivity trend analysis,
   Modular system architecture, allowing stand-alone or PACS-integrated use, with support for offline reprocessing and advanced AI-driven analysis,
   The implementation of biologically adaptive therapy,
   Generation of alerts, dose adjustment triggers, and integration with treatment planning systems.
Representative clinical applications and functional outputs may include:
   Detection of treatment response patterns, based on lack of expected conductivity response in tumors,
   Validation of targeting accuracy, particularly in SBRT, proton therapy, and sensitive anatomical regions,
   Monitoring of organs-at-risk, with customized thresholds and biological effect tracking,
   Estimation of Bragg peak, through quantification of conductivity drop-off in proton or heavy-ion therapies,
   Support for functional differentiation (e.g., necrosis vs. inflammation), aiding timing of follow-up imaging or intervention.
In some embodiments the system is configured for modular expansion and iterative optimization based on clinical datasets, supporting:
   Custom ROI definition and automated trend analysis,
   Interoperability with third-party TPS platforms,
   And eventual deployment of AI-guided biologically adaptive treatment support tools, while preserving full clinician oversight.
This integrated approach, exemplifying some embodiments of the invention, is configured to facilitate a paradigm shift in radiation therapy—moving from geometry-based verification to biological response-based treatment personalization—with broad clinical and operational implications.

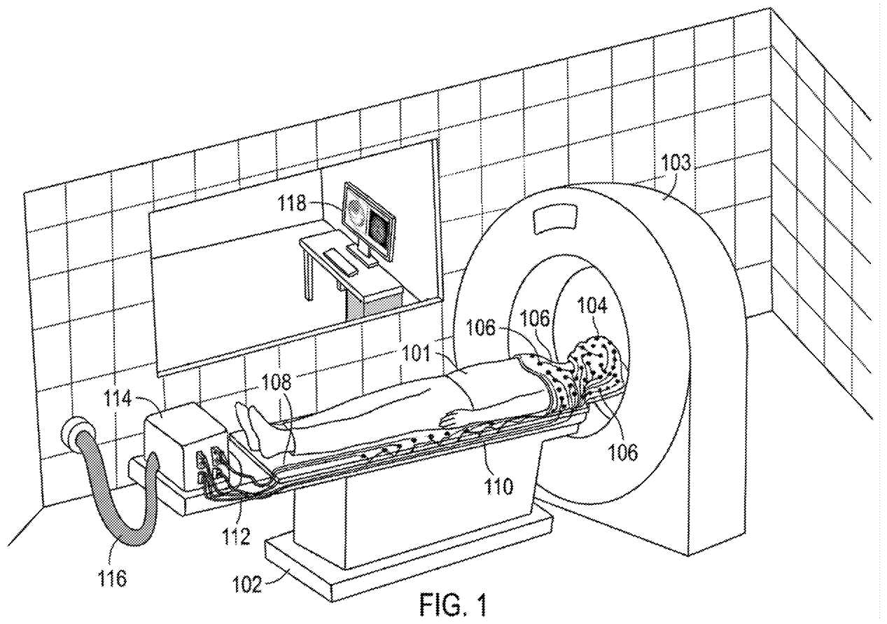

A system set up for biologically-guided adaptive radiation therapy during a simulation or treatment phase according to some embodiments is depicted in FIG. 1. As shown, patient [101] is positioned on diagnostic imaging table [102] imaging system [103] (e.g., a CT or MRI simulator bed). To ensure high-precision immobilization required for radiotherapy, patient [101] is fitted with a specialized thermoplastic head and neck mold [104].
Consistent with the “radiolucent” requirements of the present invention, mold [104] includes an array of integrated carbon-based electrodes [106] and corresponding carbon-fiber conductive wires [108]. These carbon-based components are specifically selected for their low atomic number, ensuring they do not produce significant metal artifacts or beam attenuation during anatomical imaging or radiation delivery.
The system further comprises specialized treatment tabletop [110] supported by diagnostic imaging table [102], which, in the illustrated embodiment, also features integrated electrodes to provide posterior electrical contact with patient [101] in regions not covered by mold [104].
The electrodes from both the mold and the tabletop are electrically coupled via carbon-based signal cables [112] to electrical impedance tomography (EIT) electronics box.
Electronics box [114], which may embody the impedance acquisition unit, performs the high-speed excitation and measurement routines necessary to capture biological response data. This data is transmitted (e.g., via through-wall connection [116]) to computing workstation [118] located outside the imaging room. Workstation [118] is configured for real-time image reconstruction, data processing, and the superimposition of conductivity maps onto anatomical imaging data acquired via imaging system [103], enabling the identification of biological responses—such as tumor hypoxia or perfusion changes—not otherwise characterized by standard CT or MRI alone.

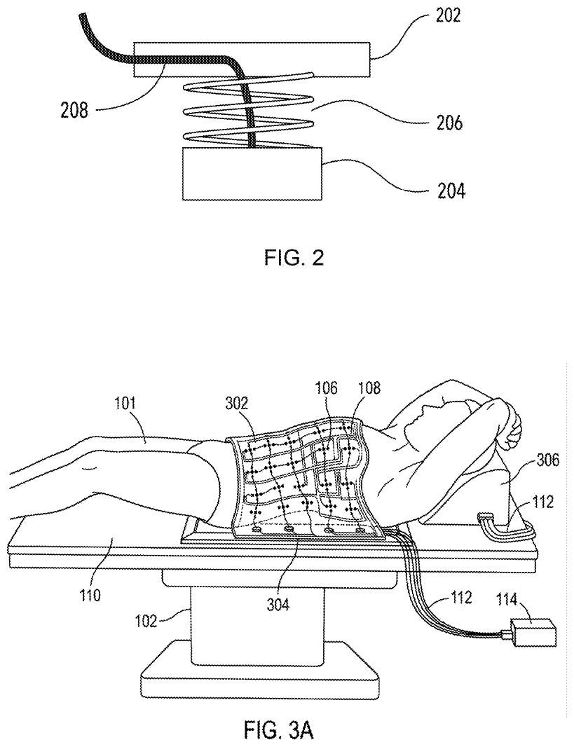

The internal architecture of the sensing hardware is integrated directly into thermoplastic immobilization mold [202], as shown in the cross-sectional view of FIG. 2. Carbon-based electrode [204] is supported by elastic element [206] to ensure stable, continuous electrical contact with the skin of patient [101] throughout the duration of a radiotherapy session.
To maintain the radiological integrity of the treatment, elastic element [206] (e.g., a spring) is comprised of a low Water Equivalent Thickness (WET) material. This selection of material ensures that the mechanical biasing of electrode [204] does not introduce significant beam attenuation or dose perturbations.
Signal connectivity is maintained through carbon-fiber conductive wire [208] that is electrically coupled to electrode [204]. As illustrated, conductive wire [208] is at least partially embedded within thermoplastic material [202]. This integrated routing protects conductive wire [208] from mechanical stress during the molding process and ensures that the electrical pathways remain fixed relative to the structural frame of the mold, even as the mold is heated and shaped to the patient's anatomy.

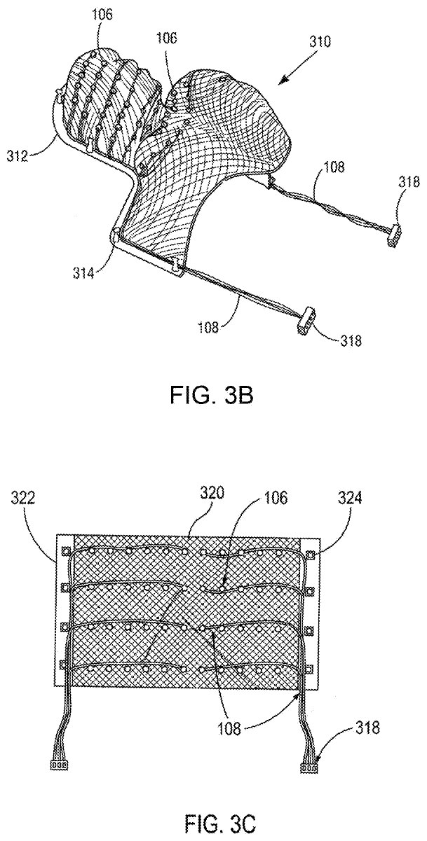

The integration of the immobilization and sensing hardware for thoracic or abdominal applications is illustrated in the perspective view of FIG. 3A. As shown, patient [101] is positioned on tabletop [110] in a supine position with arms elevated, a common posture for accessing the thoracic and pelvic regions. Tabletop [110] may be configured as a removable overlay or integrated component disposed upon diagnostic imaging table [102] or a radiotherapy treatment couch.
To ensure reproducible positioning, a thermoplastic mold [302] (configured for the thorax/abdomen) is fixated to tabletop [110] via fixation frame [304]. Thermoplastic mold [302] includes array of embedded carbon-based electrodes [106] and conductive wires [108] configured to acquire biological signals from the anterior surface of the patient's torso.
The assembly may further include headrest [306] positioned at the superior end of tabletop [110]. In the illustrated embodiment, the headrest [306] also features integrated carbon-based electrodes [106] and conductive wires [108]. While these electrodes may remain inactive during thoracic procedures, their presence allows the same hardware base to be used for multiple clinical indications without re-tooling.
Electrical connectivity for the entire assembly is provided by a plurality of signal cables [112] extending from thermoplastic mold [302] and headrest [306]. These cables are configured to interface electrical impedance tomography (EIT) electronics box [114].

Contoured thermoplastic mold assembly [310] specifically configured for head and neck immobilization is illustrated in FIG. 3B. In the depicted embodiment, contoured thermoplastic mold [310] is shown in a post-molding state, having been shaped to conform to specific anatomy of patient [101].
Integrated within thermoplastic mold assembly [310] is an array of carbon-based electrodes [106] and carbon-based wires [108]. Electrodes [106] maintain a spatial distribution along the anatomical curvature of mold [310], providing multi-angle electrical interface with head and neck regions.
Structural support and immobilization are provided by mold frame [312], which borders periphery of the thermoplastic material. Mold frame [312] includes plurality of frame clips [314] configured for secure fixation to tabletop [110] or a dedicated headrest support.
Signal transmission is facilitated by carbon-based wires [108] which exit contoured thermoplastic mold [310] and terminate at connectors [318]. These connectors [318] are configured to electrically couple mold assembly [310] to EIT electronics box [114], enabling high-frequency impedance measurements to be acquired from patient's head and neck regions during radiotherapy or imaging session.

Planar thermoplastic mold assembly [320] is illustrated in FIG. 3C in a pre-molding configuration. In this demonstrative embodiment, planar thermoplastic mold assembly [320] maintains a substantially rectangular profile intended for subsequent shaping to anatomical regions such as thorax, abdomen.
Integrated within planar thermoplastic mold assembly [320] is a grid-like array of carbon-based electrodes [106] interconnected by carbon-based wires [108]. In this embodiment in the pre-molding state, carbon-based electrodes [106] are arranged in an essentially regular, equidistant pattern across the surface of the thermoplastic material, ensuring comprehensive sensor coverage once the material is stretched and contoured to the patient. However, in some embodiments, electrodes [106] are arranged in a non-equidistant pattern. Examples of such non-equidistant distributions include:
   Increasing electrode density over specific regions of interest (ROIs), such as the prostate, liver, or tumor site, to enhance localized resolution;
   Concentrating electrodes in areas of the thermoplastic material expected to undergo high degrees of mechanical stretching—such as over the ribs or iliac crests—to ensure a more uniform distribution is achieved after the mold is shaped; or
   Offsetting electrode placement to accommodate anatomical surface irregularities or to avoid interference with other immobilization hardware.
This may be in alignment with high diagnostic priority for specific body parts, or to adjust to geometrical constraints affecting EIT functionality, such as anatomical variations or the specific volumetric reconstruction requirements of a given clinical indication.
Structural periphery is defined by mold frame [322], which supports the thermoplastic material and includes plurality of frame clips [324] for fixation to tabletop [110]. Similar to the head and neck configuration, carbon-based wires [108] are routed internally or along the surface of the mold to terminate at connectors [318]. These connectors [318] provide the electrical interface to EIT electronics box [114], allowing the system to verify electrode integrity and baseline impedance values even before the mold is custom-fitted to the patient.

Perspective view of tabletop [110] according to some embodiments, comprising integrated sensing hardware is illustrated in FIG. 3D. Tabletop [110] is configured to provide posterior electrical interface with patient [101], for example, for anatomical regions not adequately covered by a single thermoplastic mold.
Integrated within tabletop [110] is an array of carbon-based electrodes [106] and carbon-based wires [108]. Electrodes [106] are distributed across the contact surfaces of tabletop [110] to ensure contact with the patient's back and posterior-lateral torso. Similar to the mold assemblies, carbon-based wires [108] terminate at connectors [318] for electrical coupling with EIT electronics box [114].
For purposes of FIG. 3D, contact surface of tabletop [110] refers to the primary hardware surface configured to interface with patient [101]. While the integrated sensing hardware of tabletop [110] provides a stable posterior electrical interface in specific embodiments, the primary sensing interface of the system is comprised of the flexible thermoplastic mold assemblies. In some embodiments, the flexible thermoplastic mold assemblies are utilized for posterior anatomical regions even when patient [101] is in a supine position, wherein at least a portion of the flexible mold is effectively sandwiched between the posterior surface of patient [101] and the surface of imaging table [102]. Such configurations allow the flexible sensors to maintain superior conformability to the specific anatomical contours of patient [101] compared to a rigid contact plane, particularly when patient [101] is in a standing, seated, or laterally recumbent position. In yet other embodiments, tabletop [110] comprises a deformable substrate, such as a flexible foam, gel, or segmented assembly. In such configurations, the integrated sensing hardware within tabletop [110] is capable of conforming to the posterior anatomical contours of patient [101] in a manner similar to the flexible thermoplastic molds described herein. This allows the contact plane to maintain continuous electrical interface even across irregular surface geometries, effectively providing the benefits of a customized mold within a permanent tabletop structure.
Tabletop [110] further comprises plurality of fixation apertures [326]. These fixation apertures are distributed along the lateral peripheries of tabletop [110] and are specifically configured to receive and secure frame clips such as [314] and [324] of the thermoplastic mold assemblies. This mechanical integration ensures that the anterior electrodes of the mold and the posterior electrodes of tabletop [110] maintain essentially fixed, reproducible spatial relationship relative to one another and to the patient.

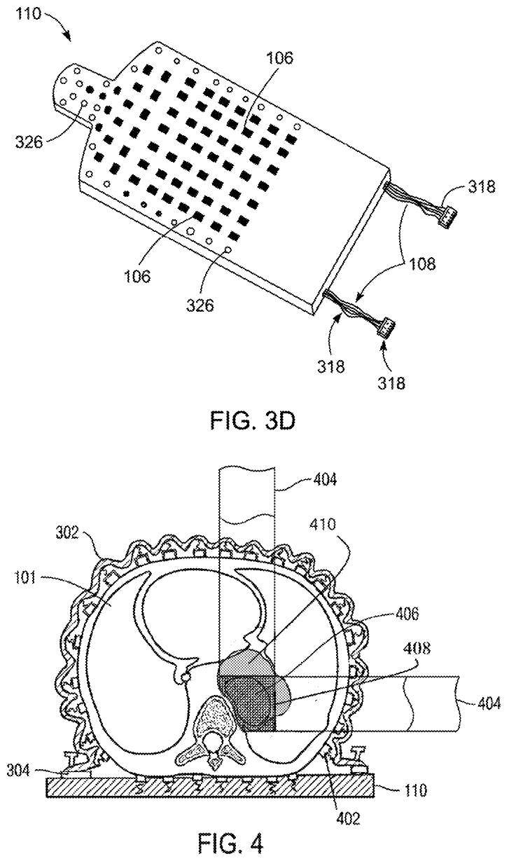

The interaction between the sensing and immobilization assembly and the patient's internal anatomy during a treatment or simulation session is illustrated in the cross-sectional view of FIG. 4. As shown, patient [101] is supported by tabletop [110], which may comprise a carbon-fiber structure with integrated carbon-based electrodes and wires. The patient body is being immobilized by thermoplastic mold [302] and tabletop [110] to which thermoplastic mold [302] is anchored via fixation frame [304]. The immobilization of the patient body serves to fix the location of target organs or tissues as much as possible during and across treatment sessions. This enhances precise spatial co-registration between the anatomical target and the treatment beam coordinates, ensuring the radiation dosage is concentrated within the tumor volume while minimizing exposure to adjacent critical structures. Furthermore, a plurality of spring-loaded carbon-fiber electrodes [402] are distributed across both mold [302] and tabletop [110] surfaces to ensure continuous, low-impedance skin contact across irregular anatomical contours.
The clinical application of the system is demonstrated by the delivery of radiation beams [404] (e.g., protons or heavy ions). In this example, two radiation beams [404] are incident at approximately 90° to one another, converging within a target tumor region [406].
The overlapping irradiated fields [408] represent the volume of primary tissue interaction with the radiation beams [404]. The depicted system is specifically configured to isolate and detect a dynamic region of maximum electrical change [410] resulting from the cumulative biological response. As illustrated, this region of maximum electrical change [410] is situated within the target tumor region [406] and extends into and beyond the irradiated fields [408]. This broader area, which may encompass and exceed the localized target tumor [406], reflects the diffusion of biological signals—such as pH shifts, ion concentration changes and gradients, cell membrane permeability, and changes in perfusion—into the surrounding normal tissue. Furthermore, this extension beyond the primary irradiated fields [408] enables the monitoring of secondary physiological responses, such as the radiation-induced bystander effect, where biological signaling propagates into non-irradiated adjacent tissue, allowing for dynamic monitoring of biological impact, namely real-time monitoring of the Bragg peak location and the physiological impact of the treatment via the EIT sensing hardware.

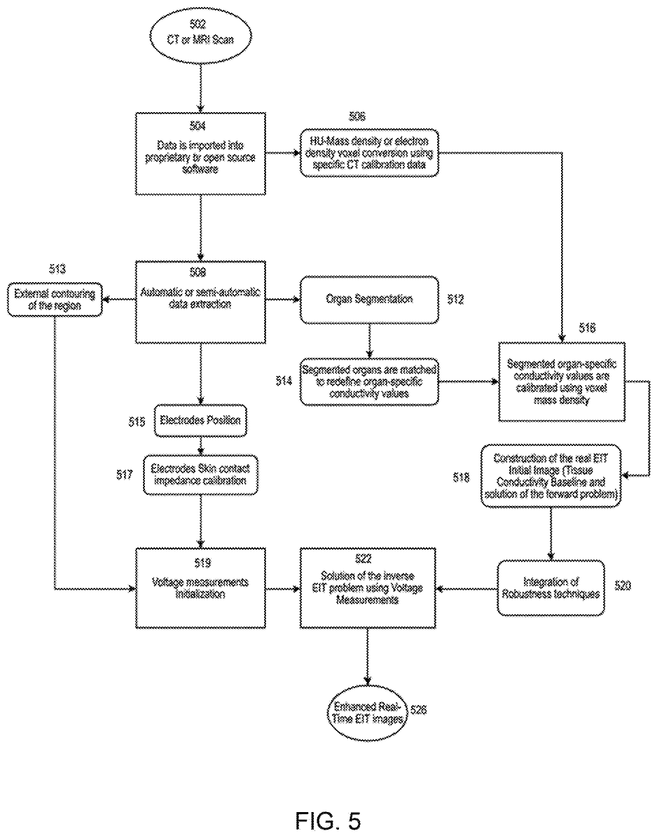

FIG. 5 illustrates a process flow diagram according to some embodiments for generating enhanced real-time EIT images through parallel anatomical and sensor-based tracks. The process initiates with a CT or MRI Scan [502]. This data is imported into proprietary or open source software [504], where a HU-Mass density or electron density voxel conversion [506] is performed using specific CT calibration data. The workflow then splits into two specialized branches:

### 1. the Anatomical and Conductivity Modeling Branch (Right)

Following automatic or semi-automatic data extraction [508], internal organs undergo AI-assisted or manual segmentation [512]. These segmented organs are matched to redefine organ-specific conductivity values [514]. Subsequently, the segmented organ-specific conductivity values are calibrated using voxel mass density [516]. Following the calibration of values in step [516], the workflow proceeds to the construction of the real EIT initial image [518], which establishes the tissue conductivity baseline and the solution of the forward problem. Subsequently, the process includes the integration of robustness techniques [520] to refine the model. These refined parameters are then utilized during the solution of the inverse EIT problem [522] using live voltage measurements (discussed below) to generate enhanced real-time EIT images [526].

### 2. the Sensor and Electrode Initialization Branch (Left)

Simultaneously, the system performs external contouring of target region [513] and defines the electrodes position [515], which is followed by an electrodes skin contact impedance calibration [517]. The external contouring of the region [513] and the electrodes skin contact impedance calibration [517] are conditional to the voltage measurements initialization [519].

### Convergence and Real-Time Output

The voltage measurements are processed along with the robust real EIT initial image through the solution of the inverse EIT problem [522] to generate enhanced real-time EIT images [526].

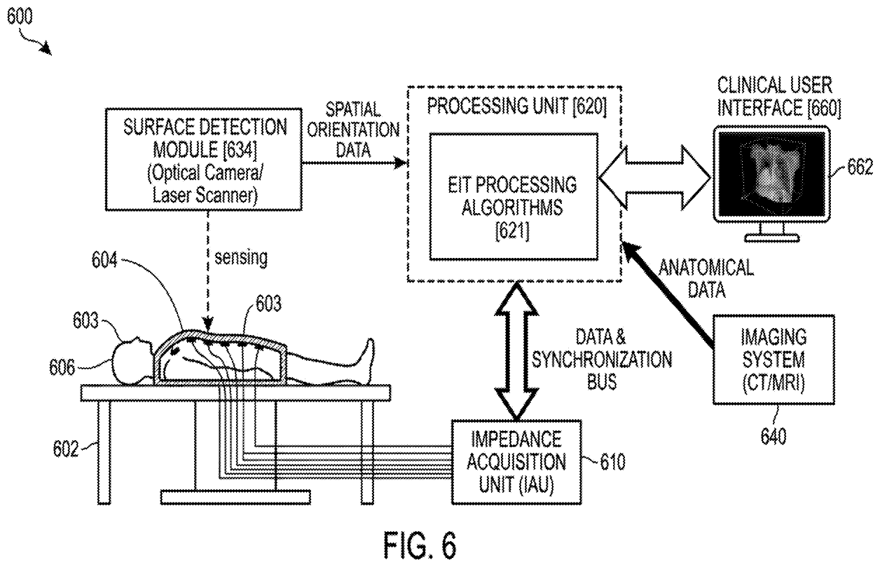

FIG. 6 illustrates a system architecture [600] according to some embodiments for a biologically-guided electrical impedance tomography (EIT) system designed to monitor biological responses in real-time by integrating anatomical priors with functional impedance data.

### Patient Interface and Hardware

Patient [606] is depicted laying supine on diagnostic imaging table [602]. To ensure spatial consistency during treatment or monitoring, an immobilization mold [604] may be employed. Radiolucent electrodes [603] are integrated into mold [604] and in contact with the patient's skin within the target anatomical region.

### Data Acquisition and Sensing

The radiolucent electrodes [603] are electrically coupled to an Impedance Acquisition Unit (IAU) [610]. The IAU [610] manages the injection of alternating current and the measurement of resulting boundary voltages. Simultaneously, a Surface Detection Module [634], which may include an optical camera or laser scanner, performs “sensing” of the patient's exterior. This module identifies the spatial orientation of electrodes [603] relative to the patient's body.

### The Processing Unit and Software Logic

Processing Unit [620] receives spatial orientation data from Surface Detection Module [634] and anatomical data from Imaging System [640] (such as a CT or MRI) and is configured to execute the following functions through Processing Algorithms [621]:
   Spatially co-register of the input received from Surface Detection Module [634] and Imaging System [640] to define a digital volumetric mesh corresponding to the target anatomical region.
   Reconstruct one or more volumetric conductivity maps from data provided by IAU by mapping this data onto the volumetric mesh
   Superimpose the volumetric conductivity map onto the anatomical imaging data to identify biological responses

### Baseline Comparison and Clinical Output

The depicted system is configured to establish a patient-specific conductivity baseline derived from pre-treatment measurements. During or between treatment fractions, subsequent volumetric conductivity maps are generated. Processing Unit [620] determines a “conductivity change” by comparing these active maps against the stored baseline.

### Clinical User Interface

The volumetric conductivity map superimposed onto the anatomical imaging data—is displayed through monitor [662] by Clinical User Interface [660] communicatively coupled to Processing Unit [620]. This visualization on the monitor [662] facilitates the identification of the biological responses such as inflammation, edema, or tumor displacement).

It should be understood that elements and/or features of an apparatus, or a method described herein can be combined in a variety of ways without departing from the spirit and scope of the present teachings, whether explicit or implicit herein. For example, where reference is made to a particular structure, that structure can be used in various embodiments of apparatus of the present teachings and/or in methods of the present teachings, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.
The use of the singular herein, for example, “a,” “an,” and “the,” includes the plural (and vice versa) unless specifically stated otherwise.
The use of any and all examples, or exemplary language herein, for example, “such as,” “including,” or “for example,” is intended merely to better illustrate the present teachings and does not pose a limitation on the scope of the invention unless claimed. No language in the specification should be construed as indicating any non-claimed element as essential to the practice of the present teachings.
The present teachings encompass embodiments in other specific forms without departing from the spirit or essential characteristics thereof. The foregoing embodiments are therefore to be considered in all respects illustrative rather than limiting on the present teachings described herein.

1. A biologically-guided monitoring and treatment assistance system, comprising:
a data interface configured to receive signals from a plurality of radiolucent electrodes;
a surface detection module configured to identify a spatial orientation of said plurality of radiolucent electrodes relative to a target anatomical region of a patient;
an impedance acquisition unit coupled to said plurality of radiolucent electrodes;
and a processing unit configured to:
(i) generate a spatial co-registration of said spatial orientation from said surface detection module with anatomical imaging data of the target anatomical region to define a digital volumetric mesh;
(ii) reconstruct at least one volumetric conductivity map from data provided by said impedance acquisition unit by mapping said data onto said volumetric mesh; and
(iii) superimpose said volumetric conductivity map onto said anatomical imaging data to identify at least one biological response not characterized by the anatomical imaging data.

2. The system of claim 1, further comprising at least one immobilization interface comprising a thermoplastic material and a plurality of radiolucent electrodes electrically coupled to said impedance acquisition unit, wherein said plurality of radiolucent electrodes is embedded within the thermoplastic material of said immobilization interface, and wherein at least one of said electrodes from said plurality of radiolucent electrodes is coupled to a radiolucent biasing element configured to maintain constant physical contact between said electrode coupled to biasing element and the patient skin.

3. The system of claim 1, wherein the processing unit is further configured to synchronize reconstruction of said at least one volumetric conductivity map with a radiation delivery schedule of a radiotherapy treatment beam.

4. The system of claim 1, wherein said processing unit comprises a forward modeling module configured to generate a patient-specific conductivity distribution using segmented anatomical imaging data and organ-specific conductivity values;
said processing unit being further configured to perform an inverse reconstruction of EIT data using said forward modeling module as an anatomical prior to generate:
(a) a patient-specific conductivity baseline derived from a pre-treatment measurement; and
(b) subsequent volumetric conductivity maps during or between one or more treatment fractions;
wherein the processing unit is configured to determine a conductivity change by comparing said subsequent maps to said patient-specific conductivity baseline

5. The system of claim 1, wherein the processing unit comprises an AI-based module configured to translate reconstructed EIT images into synthetic anatomical representations using a trained image-to-image deep learning model.

6. The system of claim 1, wherein said processing unit is further configured to store a plurality of temporal reconstructed volumetric conductivity maps and identify temporal trends in biological response by comparing conductivity values across said plurality of reconstructed volumetric conductivity maps.

7. The system of claim 6, wherein at least one of said maps is reconstructed following ionizing radiation delivery and said identification of said temporal trends associated with conductivity changes induced by said ionizing radiation delivery.

8. The system of claim 1, wherein said processing unit is further configured to verify the beam range of a particle therapy beam by identifying a localized increase in conductivity corresponding to a Bragg peak location.

9. The system of claim 1, wherein said processing unit is further configured to generate a clinical alert when a change in said at least one volumetric conductivity map exceeds a predefined threshold.

10. The system of claim 1, wherein said processing unit is further configured to export data derived from said at least one volumetric conductivity map to a treatment planning system (TPS) for recalculation of a radiation dose.

11. The system of claim 1, further comprising a patient support assembly having a tabletop, wherein at least a portion of said tabletop is radiolucent and comprises a plurality of said radiolucent electrode.

12. The system of claim 1, further comprising at least one internal electrode integrated into a catheter needle or a brachytherapy applicator, wherein said processing unit is configured to reconstruct said volumetric conductivity maps using electrical impedance measurements generated between said internal electrode and at least one of said plurality of radiolucent electrodes.

13. A method for biologically-guided monitoring and treatment assistance, comprising:
receiving, at a processing unit, impedance data originating from a plurality of radiolucent electrodes;
identifying, via a surface detection module, a spatial orientation of said plurality of radiolucent electrodes relative to a target anatomical region of a patient;
collecting impedance data from the target anatomical region via an impedance acquisition unit coupled to said plurality of radiolucent electrodes;
generating, via a processing unit, a spatial co-registration of said spatial orientation with anatomical imaging data of the target anatomical region to define a digital volumetric mesh;
reconstructing at least one volumetric conductivity map by mapping said collected impedance data onto said volumetric mesh; and
identifying at least one biological response not characterized by the anatomical imaging data by superimposing said at least one volumetric conductivity map onto said anatomical imaging data.

14. The method of claim 13, further comprising securing at least one immobilization interface comprising said plurality of radiolucent electrodes to the patient.

15. The method of claim 13, further comprising synchronizing, via the processing unit, the reconstruction of said at least one volumetric conductivity map with a radiation delivery schedule of a radiotherapy treatment beam.

16. The method of claim 13, further comprising:
(a) generating, via the processing unit, a patient-specific initial conductivity distribution using a forward modeling module and segmented anatomical imaging data with organ-specific conductivity values;
(b) performing, via the processing unit, an inverse reconstruction of EIT data using said forward modeling module as an anatomical prior to incorporate anatomical imaging data as spatial and electrical priors; and
(c) generating, via the processing unit, a patient-specific conductivity baseline using a baseline conductivity model derived from said anatomical imaging data and organ-specific conductivity references;
wherein said reconstructing includes generating subsequent volumetric conductivity maps and comparing them to said patient-specific conductivity baseline.

17. The method of claim 13, further comprising translating, via an AI-based module, reconstructed electrical impedance tomography (EIT) images into synthetic anatomical representations by applying a trained image-to-image deep learning model to said reconstructed images.

18. The method of claim 13, further comprising:
storing a plurality of temporal reconstructed volumetric conductivity maps; and
identifying temporal trends in biological response by comparing conductivity values across said plurality of temporal reconstructed volumetric conductivity maps.

19. The method of claim 18, wherein:
at least one of said plurality of temporal reconstructed volumetric conductivity maps is reconstructed following ionizing radiation delivery; and
said identifying of said temporal trends comprises identifying conductivity changes induced by said ionizing radiation delivery.

20. The method of claim 13, further comprising verifying, via the processing unit, a beam range of a particle therapy beam by identifying a localized increase in conductivity corresponding to a Bragg peak location.

21. The method of claim 13, further comprising generating, via the processing unit, a clinical alert when a change in said at least one volumetric conductivity map exceeds a predefined threshold.

22. The method of claim 13, further comprising:
exporting data derived from said at least one volumetric conductivity map to a treatment planning system (TPS); and
recalculating a radiation dose based on said exported data.

23. The method of claim 13, further comprising providing a patient support assembly having a tabletop, wherein at least a portion of said tabletop is radiolucent and comprises at least one of said plurality of radiolucent electrodes.

24. The method of claim 13, further comprising integrating at least one internal electrode into a catheter needle or a brachytherapy applicator, and reconstructing said volumetric conductivity maps using electrical impedance measurements generated between said internal electrode and at least one of said plurality of radiolucent electrodes.

25. A non-transitory computer-readable medium storing instructions that, when executed by a processing unit, cause said processing unit to perform a method for biologically-guided monitoring and treatment assistance, the method comprising:
receiving a spatial orientation of a plurality of radiolucent electrodes relative to a target anatomical region of a patient from a surface detection module;
receiving impedance data from an impedance acquisition unit coupled to said plurality of radiolucent electrode;
generating a spatial co-registration of said spatial orientation with anatomical imaging data of the target anatomical region to define a digital volumetric mesh;
reconstructing at least one volumetric conductivity map by mapping the received impedance data onto said volumetric mesh; and
superimposing said at least one volumetric conductivity map onto said anatomical imaging data to identify at least one biological response not characterized by the anatomical imaging data.

26. A radiolucent immobilization interface for use in a biologically-guided radiotherapy monitoring system, comprising:
radiolucent substrate configured to conform to a target anatomical region of a patient and maintain said target anatomical region in a fixed position relative to a radiation delivery source;
a plurality of radiolucent electrodes integrated into or disposed upon said substrate, wherein said electrodes are positioned to establish electrical contact with the skin of the patient when the substrate is in an immobilization position; and
a plurality of radiolucent conductive leads coupled to said plurality of radiolucent electrodes, said leads extending to an interface connector configured for electrical coupling to an external impedance acquisition unit.

27. The interface of claim 26, wherein said radiolucent substrate comprises a thermoplastic material configured to be molded to the patient's specific anatomical contours and to retain said contours upon cooling.

28. The interface of claim 26, wherein at least one electrode of said plurality of radiolucent electrodes is coupled to a radiolucent biasing element configured to maintain constant physical contact between said electrode and the patient's skin.
